(19) United States
(12) Patent Application Publication
(10) Pub. No.: US 2023/0086755 A1
(43) Pub. Date: Mar. 23, 2023

(54) REAGENT COMPOSITIONS, METHODS, CARTRIDGES, AND SYSTEMS

(71) Applicants: ILLUMINA CAMBRIDGE LIMITED, Cambridge (GB); ILLUMINA SINGAPORE PTE. LTD., Singapore (SG)

(72) Inventors: Jessica WALSH, Cambridge (GB); Sébastien RICOULT, Cambridge (GB); Yin Nah TEO, Singapore (SG); Simon BAKER, Cambridge (GB)

(73) Assignees: ILLUMINA CAMBRIDGE LIMITED, Cambridge (GB); ILLUMINA SINGAPORE PTE. LTD., Singapore (SG)

(21) Appl. No.: 17/932,709

(22) Filed: Sep. 16, 2022

Related U.S. Application Data
(60) Provisional application No. 63/245,467, filed on Sep. 17, 2021.

(51) Int. Cl. C12Q 1/6869; C12Q 1/6806

(52) U.S. Cl. CPC C12Q 1/6869; C12Q 1/6806

(57) ABSTRACT

The present disclosure relates to compositions including a shell surrounding an interior compartment, wherein said interior compartment comprises one or more reagent and wherein said shell releases said interior compartment when said shell is exposed to a first release condition, wherein said interior compartment releases said one or more reagent when said interior compartment is exposed to a second release condition, and wherein said first release condition is different from said second release condition. Also disclosed are compositions including a dissolvable first shell, and a dissolvable second shell, the second shell comprising one or more reagent. Also disclosed are methods for controlling release of one or more reagent using the compositions described herein. The present disclosure further relates to cartridges that include a reagent reservoir including the compositions described herein. Also disclosed are systems for controlling release of one or more reagent including the compositions described herein.

### CROSS-REFERENCE TO RELATED APPLICATIONS

This application claims benefit of U.S. Provisional Patent Application Ser. No. 63/245,467, filed Sep. 17, 2021, which is hereby incorporated by reference in its entirety.

### FIELD

The present disclosure relates generally to compositions, methods, cartridges, and systems for controlling release of reagents.

### BACKGROUND

Many current sequencing platforms use “sequencing by synthesis” (“SBS”) technology and fluorescence-based methods for detection. Alternative sequencing methods and improved sample and library preparation processes that allow for more cost effective, rapid, and convenient sequencing and nucleic acid detection are desirable as complements to SBS.

Current protocols for SBS technology routinely employ a sample preparation process that converts DNA or RNA into a library of fragmented templates suitable for sequencing. Sample preparation methods often involve multiple steps, material transfers, and expensive instruments to effect fragmentation, and, therefore, are often difficult, tedious, expensive, and inefficient.

Libraries including polynucleotides are generally prepared in any suitable manner to attach oligonucleotide adapters to target polynucleotides. Sequencing may result in determination of the sequence of the whole, or a part of the target polynucleotides. The number of steps involved to transform nucleic acids into adapter-modified templates in solution ready for cluster formation and sequencing can be reduced, or in some instances even minimized, by the use of transposase mediated fragmentation and tagging. This process, referred to as “tagmentation,” involves the modification of nucleic acids by a transposome complex comprising transposase enzyme complexed with adapters comprising transposon end sequence, as described in, for example, WO 2016/130704. Methods for immobilizing and amplifying prior to sequencing are described in, for instance, U.S. Pat. No. 8,053,192. A library of templates may be used to prepare clustered arrays of nucleic acid colonies, as described in U.S. Pat. Publ. No. 2005/0100900, by solid-phase amplification and more particularly solid phase isothermal amplification.

Sequencing can be carried out using any suitable sequencing technique, and methods for determining the sequence of immobilized and amplified adapter-target-adapter molecules, including strand re-synthesis, are known in the art and are described in, for instance, U.S. Pat. No. 8,053,192. SBS techniques generally involve the enzymatic extension of a nascent nucleic acid strand through the iterative addition of nucleotides against a template strand. In traditional methods of SBS, a single nucleotide monomer may be provided to a target nucleotide in the presence of a polymerase in each delivery. Exemplary SBS systems and methods are described in U.S. Pat. Publ. No. 2007/0166705.

There are various problems that block efficiency of sample preparation compositions and processes for sequencing. For example, there are difficulties in staggering dissolution of multiple reagents within a common well. There are problems of differentiating between different reagents in terms of dissolving time. There are also problems of purifying atmospheric-captured water for sample and library preparation compositions and processes for sequencing.

Accordingly, there is a need for improved sample preparation compositions and processes. In particular, there is a need for sequencing reagents, sample preparation reagents, and library preparation reagents with improved stability and associated compositions, methods, cartridges, and systems that demonstrate improved efficiency of workflow and tagmented library production and, in turn, increased read enrichment for the resulting libraries and simplified workflows.

The present disclosure is directed to overcoming these and other deficiencies in the art.

### SUMMARY

A first aspect relates to a composition. The composition includes a shell surrounding an interior compartment, wherein said interior compartment comprises one or more reagent and wherein said shell releases said interior compartment when said shell is exposed to a first release condition, wherein said interior compartment releases said one or more reagent when said interior compartment is exposed to a second release condition, and wherein said first release condition is different from said second release condition.

In one implementation, the interior compartment prevents release of the one or more reagent when the shell is exposed to the first release condition. In one implementation, the first release condition occurs before the second release condition. In another implementation, the second release condition occurs after the first release condition.

In one implementation, the first release condition comprises a temperature-controlled release condition, a pH-controlled release condition, a time-controlled release condition, a position-controlled release condition, or any combination thereof. In another implementation, the second release condition comprises a temperature-controlled release condition, a pH-controlled release condition, a time-controlled release condition, a position-controlled release condition, or any combination thereof. In one implementation, either or both of the first and second release conditions comprise a change in temperature. In another implementation, the change in temperature is to a temperature above about 25° C. In another implementation, the change in temperature is to a temperature at or below about 25° C.

In one implementation, the shell releases the interior compartment when the shell is exposed to at least one additional shell release condition, where one or more of the at least one additional shell release condition is different from the first release condition. In one implementation, the interior compartment prevents release of the one or more reagent when the shell is exposed to the at least one additional shell release condition. In another implementation, the interior compartment releases the one or more reagent when the interior compartment is exposed to at least one additional interior compartment release condition, where one or more of the at least one additional interior compartment release condition is different from the second release condition.

In one implementation, the shell has a shell width and the interior compartment has an interior compartment width, and the shell width is different from the interior compartment width. In one implementation, the shell width is between about 1 micrometer and about 1,000 micrometers. In another implementation, the interior compartment width is between about 1 micrometer and about 1,000 micrometers.

In one implementation, the shell comprises a water-soluble compound. In one implementation, the shell comprises one or more of polyvinyl alcohol, polyvinylpyrrolidone (PVP), carrageenan, gelatin, hydroxypropyl methylcellulose (HPMC), pullulan, starch film, benzoxaborole-poly(vinyl alcohol) (benzoxaborole-PVA), pectin, or any combination thereof.

In one implementation, the one or more reagent is a sequencing reagent, a sample preparation reagent, a library preparation reagent, or a combination thereof. In one implementation, the one or more reagent is selected from one or more enzyme, salt, surfactant, buffering agent, enzyme inhibitor, primer, nucleotide, organic osmolite, magnetic bead, molecular probe, crowding agent, small molecule, labelled-nucleotide, or any combination thereof.

In one implementation, the composition further comprises a water purification compound. In one implementation, the water purification compound comprises sodium dichloroisocyanurate, chlorine, chloramines, chlorine dioxide, polyaluminium chloride, aluminum sulfate, ferric sulfate, hydrogen peroxide, sodium hydroxide bromide, silver nanoparticles, iron, iodine, activated carbon, or any combination thereof.

In one implementation, the interior compartment comprises one or more dry reagent, one or more microsphere, one or more bead, one or more powder, one or more cake, one or more gel, one or more liquid, or any combination thereof. In one implementation, the one or more reagent is lyophilised. In one implementation, the interior compartment comprises a plurality of microspheres comprising a plurality of reagents. In another implementation, the interior compartment comprises a plurality of microspheres comprising one reagent.

A second aspect relates to a composition. The composition includes a dissolvable first shell, and a dissolvable second shell, the second shell comprising one or more reagent.

In one implementation, the first shell is an exterior shell. In one implementation, the second shell is an interior shell. In one implementation, the first shell dissolves when the composition is exposed to a first release condition. In one implementation, the second shell prevents release of the one or more reagent when the composition is exposed to the first release condition. In another implementation, the second shell dissolves when exposed to a second release condition.

In one implementation, the first release condition comprises a temperature-controlled release condition, a pH-controlled release condition, a time-controlled release condition, a position-controlled release condition, or any combination thereof. In another implementation, the second release condition comprises a temperature-controlled release condition, a pH-controlled release condition, a time-controlled release condition, a position-controlled release condition, or any combination thereof. In one implementation, either or both of the first and second release conditions comprise a change in temperature. In another implementation, the change in temperature is to a temperature above about 25° C. In yet another implementation, the change in temperature is to a temperature at or below about 25° C.

In one implementation, the first shell dissolves when the first shell is exposed to at least one additional first shell release condition, where one or more of the at least one additional first shell release condition is different from the first release condition. In another implementation, the second shell prevents release of the one or more reagent when the second shell is exposed to the at least one additional first shell release condition. In one implementation, the second shell releases the one or more reagent when the second shell is exposed to at least one additional second shell release condition, where one or more of the at least one additional second shell release condition is different from the second release condition.

In one implementation, the first shell has a first shell width and the second shell has a second shell width, and the first shell width is different from the second shell width. In another implementation, the first shell width is between about 1 micrometer and about 1,000 micrometers. In yet another implementation, the second shell width is between about 1 micrometer and about 1,000 micrometers.

In one implementation, the first shell comprises a water-soluble compound. In another implementation, the first shell comprises one or more of polyvinyl alcohol, polyvinylpyrrolidone (PVP), carrageenan, gelatin, hydroxypropyl methylcellulose (HPMC), pullulan, starch film, benzoxaborole-poly(vinyl alcohol) (benzoxaborole-PVA), pectin, or any combination thereof.

In one implementation, the one or more reagent is a sequencing reagent, a sample preparation reagent, a library preparation reagent, or a combination thereof. In another implementation, the one or more reagent is selected from one or more enzyme, salt, surfactant, buffering agent, enzyme inhibitor, primer, nucleotide, organic osmolite, magnetic bead, molecular probe, crowding agent, small molecule, labelled-nucleotide, or any combination thereof.

In one implementation, the composition further comprises a water purification compound. In one implementation, the water purification compound comprises sodium dichloroisocyanurate, chlorine, chloramines, chlorine dioxide, polyaluminium chloride, aluminum sulfate, ferric sulfate, hydrogen peroxide, sodium hydroxide bromide, silver nanoparticles, iron, iodine, activated carbon, or any combination thereof.

In one implementation, the second shell comprises one or more dry reagent, one or more microsphere, one or more bead, one or more powder, one or more cake, one or more gel, one or more liquid, or any combination thereof. In another implementation, the one or more reagent is lyophilised. In one implementation, the second shell comprises a plurality of microspheres comprising a plurality of reagents. In another implementation, the second shell comprises a plurality of microspheres comprising one reagent.

A third aspect relates to a composition. The composition includes a dissolvable first shell; a dissolvable second shell, the second shell comprising one or more reagent; and a water purification compound.

In one implementation, the water purification compound is in a position between the dissolvable first shell and the dissolvable second shell.

In one implementation, the water purification compound comprises sodium dichloroisocyanurate, chlorine, chloramines, chlorine dioxide, polyaluminium chloride, aluminum sulfate, ferric sulfate, hydrogen peroxide, sodium hydroxide bromide, silver nanoparticles, iron, iodine, activated carbon, or any combination thereof. In one implementation, the first shell is an exterior shell. In another implementation, the second shell is an interior shell.

A fourth aspect relates to a method for controlling release of one or more reagent. The method includes providing a composition comprising a shell surrounding an interior compartment, wherein said interior compartment comprises one or more reagent; exposing said composition to a first release condition to release said interior compartment; and exposing said interior compartment to a second release condition to release said one or more reagent, wherein said first release condition is different from said second release condition.

In one implementation, the interior compartment prevents release of the one or more reagent when the shell is exposed to the first release condition. In one implementation, the first release condition occurs before the second release condition. In another implementation, the second release condition occurs after the first release condition.

In one implementation, the first release condition comprises a temperature-controlled release condition, a pH-controlled release condition, a time-controlled release condition, a position-controlled release condition, or any combination thereof. In another implementation, the second release condition comprises a temperature-controlled release condition, a pH-controlled release condition, a time-controlled release condition, a position-controlled release condition, or any combination thereof. In one implementation, either or both of the first and second release conditions comprise a change in temperature. In another implementation, the change in temperature is to a temperature above about 25° C. In yet another implementation, the change in temperature is to a temperature at or below about 25° C.

In one implementation, the first release condition comprises a pH of between about 1.0 and about 10.0. In another implementation, the second release condition comprises a pH of between about 1.0 and about 10.0. In one implementation, the second release condition is effective to release a plurality of reagents, where the content of at least one reagent is different from the content of at least one other reagent. In one implementation, exposing the shell to the first release condition and exposing the interior compartment to the second release condition occurs sequentially.

In one implementation, the shell releases the interior compartment when the shell is exposed to at least one additional shell release condition, where one or more of the at least one additional shell release condition is different from the first release condition. In another implementation, the interior compartment prevents release of the one or more reagent when the shell is exposed to the at least one additional shell release condition.

In one implementation, the interior compartment releases the one or more reagent when the interior compartment is exposed to at least one additional interior compartment release condition, where one or more of the at least one additional interior compartment release condition is different from the second release condition. In another implementation, the shell has a shell width and the interior compartment has an interior compartment width, and the shell width is different from the interior compartment width. In another implementation, the shell width is between about 1 micrometer and about 1,000 micrometers. In yet another implementation, the interior compartment width is between about 1 micrometer and about 1,000 micrometers.

In one implementation, the shell comprises a water-soluble compound. In one implementation, the shell comprises one or more of polyvinyl alcohol, polyvinylpyrrolidone (PVP), carrageenan, gelatin, hydroxypropyl methylcellulose (HPMC), pullulan, starch film, benzoxaborole-poly(vinyl alcohol) (benzoxaborole-PVA), pectin, or any combination thereof.

In one implementation, the one or more reagent is a sequencing reagent, a sample preparation reagent, a library preparation reagent, or a combination thereof. In one implementation, the one or more reagent is selected from one or more enzyme, salt, surfactant, buffering agent, enzyme inhibitor, primer, nucleotide, organic osmolite, magnetic bead, molecular probe, crowding agent, small molecule, labelled-nucleotide, or any combination thereof.

In one implementation, the method further comprises providing a water purification compound. In one implementation, the water purification compound comprises sodium dichloroisocyanurate, chlorine, chloramines, chlorine dioxide, polyaluminium chloride, aluminum sulfate, ferric sulfate, hydrogen peroxide, sodium hydroxide bromide, silver nanoparticles, iron, iodine, activated carbon, or any combination thereof.

In one implementation, the interior compartment comprises one or more dry reagent, one or more microsphere, one or more bead, one or more powder, one or more cake, one or more gel, one or more liquid, or any combination thereof. In one implementation, the one or more reagent is lyophilised. In one implementation, the interior compartment comprises a plurality of microspheres comprising a plurality of reagents. In another implementation, the interior compartment comprises a plurality of microspheres comprising one reagent.

A fifth aspect relates to a method for controlling release of one or more reagent. The method includes providing a composition comprising: a dissolvable first shell, and a dissolvable second shell, the second shell comprising one or more reagent; exposing said composition to a first release condition to dissolve said first shell; and exposing said composition to a second release condition to dissolve said second shell, wherein said first release condition is different from said second release condition.

In one implementation, the second shell prevents release of the one or more reagent when the composition is exposed to the first release condition. In one implementation, the first release condition comprises a temperature-controlled release condition, a pH-controlled release condition, a time-controlled release condition, a position-controlled release condition, or any combination thereof. In one implementation, the second release condition comprises a temperature-controlled release condition, a pH-controlled release condition, a time-controlled release condition, a position-controlled release condition, or any combination thereof. In one implementation, either or both of the first and second release conditions comprise a change in temperature. In another implementation, the change in temperature is to a temperature above about 25° C. In yet another implementation, the change in temperature is to a temperature at or below about 25° C.

In one implementation, the first shell dissolves when the first shell is exposed to at least one additional first shell release condition, where one or more of the at least one additional first shell release condition is different from the first release condition. In another implementation, the second shell prevents release of the one or more reagent when the second shell is exposed to the at least one additional first shell release condition. In another implementation, the second shell releases the one or more reagent when the second shell is exposed to at least one additional second shell release condition, where one or more of the at least one additional second shell release condition is different from the second release condition. In one implementation, the first shell has a first shell width and the second shell has a second shell width, and the first shell width is different from the second shell width. In another implementation, the first shell width is between about 1 micrometer and about 1,000 micrometers. In yet another implementation, the second shell width is between about 1 micrometer and about 1,000 micrometers.

In one implementation, the first shell comprises a water-soluble compound. In one implementation, the first shell comprises one or more of polyvinyl alcohol, polyvinylpyrrolidone (PVP), carrageenan, gelatin, hydroxypropyl methylcellulose (HPMC), pullulan, starch film, benzoxaborole-poly(vinyl alcohol) (benzoxaborole-PVA), pectin, or any combination thereof.

In one implementation, the one or more reagent is a sequencing reagent, a sample preparation reagent, a library preparation reagent, or a combination thereof. In one implementation, the one or more reagent is selected from one or more enzyme, salt, surfactant, buffering agent, enzyme inhibitor, primer, nucleotide, organic osmolite, magnetic bead, molecular probe, crowding agent, small molecule, labelled-nucleotide, or any combination thereof.

In one implementation, the method further comprises providing a water purification compound. In one implementation, the water purification compound comprises sodium dichloroisocyanurate, chlorine, chloramines, chlorine dioxide, polyaluminium chloride, aluminum sulfate, ferric sulfate, hydrogen peroxide, sodium hydroxide bromide, silver nanoparticles, iron, iodine, activated carbon, or any combination thereof. In one implementation, the interior shell comprises one or more dry reagent, one or more microsphere, one or more bead, one or more powder, one or more cake, one or more gel, one or more liquid, or any combination thereof. In one implementation, the one or more reagent is lyophilised. In one implementation, the interior shell comprises a plurality of microspheres comprising a plurality of reagents. In another implementation, the interior shell comprises a plurality of microspheres comprising one reagent. In one implementation, the first shell is an exterior shell. In another implementation, the second shell is an interior shell.

A sixth aspect relates to a method for controlling release of one or more reagent. The method includes providing a composition comprising: a dissolvable first shell, a dissolvable second shell, the second shell comprising one or more reagent, and a water purification compound; exposing said composition to a first release condition to dissolve said water purification compound; exposing said composition to a second condition to dissolve said first shell; and exposing said composition to a third release condition to dissolve said second shell, wherein said first release condition is different from said second release condition.

In one implementation, the water purification compound comprises sodium dichloroisocyanurate, chlorine, chloramines, chlorine dioxide, polyaluminium chloride, aluminum sulfate, ferric sulfate, hydrogen peroxide, sodium hydroxide bromide, silver nanoparticles, iron, iodine, activated carbon, or any combination thereof. In one implementation, the first shell is an exterior shell. In another implementation, the second shell is an interior shell.

A seventh aspect relates to a method. The method includes providing a capsule in a well at a first temperature; providing a liquid having a temperature in said well; elevating the temperature of the liquid to a second temperature; lowering the temperature of the liquid from the second temperature to a third temperature; and releasing one or more reagents from said capsule.

In one implementation, the capsule comprises a composition comprising a shell surrounding an interior compartment, where the interior compartment comprises one or more reagent and where the shell releases the interior compartment when the shell is exposed to a first release condition, where the interior compartment releases the one or more reagent when the interior compartment is exposed to a second release condition, and where the first release condition is different from the second release condition. In one implementation, the capsule comprises the composition comprising a dissolvable first shell, and a dissolvable second shell, the second shell comprising one or more reagent. In one implementation, the capsule comprises the composition comprising a dissolvable first shell; a dissolvable second shell, the second shell comprising one or more reagent; and a water purification compound. In one implementation, the second temperature is above about 25° C. In one implementation, the third temperature is at or below about 25° C.

In one implementation the method further comprises providing a water purification compound. In one implementation, the water purification compound comprises sodium dichloroisocyanurate, chlorine, chloramines, chlorine dioxide, polyaluminium chloride, aluminum sulfate, ferric sulfate, hydrogen peroxide, sodium hydroxide bromide, silver nanoparticles, iron, iodine, activated carbon, or any combination thereof.

In one implementation, the capsule comprises a water-soluble compound. In one implementation, the capsule comprises one or more of polyvinyl alcohol, polyvinylpyrrolidone (PVP), carrageenan, gelatin, hydroxypropyl methylcellulose (HPMC), pullulan, starch film, benzoxaborole-poly(vinyl alcohol) (benzoxaborole-PVA), pectin, or any combination thereof.

In one implementation, the one or more reagent is a sequencing reagent, a sample preparation reagent, a library preparation reagent, or a combination thereof. In one implementation, the one or more reagent is selected from one or more enzyme, salt, surfactant, buffering agent, enzyme inhibitor, primer, nucleotide, organic osmolite, magnetic bead, molecular probe, crowding agent, small molecule, labelled-nucleotide, or any combination thereof.

In one implementation, the interior compartment comprises one or more dry reagent, one or more microsphere, one or more bead, one or more powder, one or more cake, one or more gel, one or more liquid, or any combination thereof. In one implementation, the one or more reagent is lyophilised. In one implementation, the interior compartment comprises a plurality of microspheres comprising a plurality of reagents. In another implementation, the interior compartment comprises a plurality of microspheres comprising one reagent. In one implementation, the first temperature is different from the third temperature. In another implementation, the first temperature is the same as the third temperature.

An eighth aspect relates to a method. The method includes dissolving an exterior shell of a capsule in a well at a first temperature, where the well comprises a liquid, where the capsule comprises the exterior shell, a water purification compound, an interior shell, and one or more reagent, where dissolving the exterior shell of the capsule releases the water purification compound; elevating the temperature of the well to a second temperature; and dissolving the interior shell thereby releasing one or more reagent.

In one implementation, dissolving the exterior shell of the capsule in the well comprises flowing the liquid into the well. In another implementation, dissolving the interior shell comprises raising the pH of the liquid above 7.0. In another implementation, dissolving the interior shell comprises lowering the pH of the liquid below 7.0. In yet another implementation, the interior shell is dissolved by the second temperature. In another implementation, the interior shell is dissolved after a minimum time period. In one implementation, the minimum time period is 5 minutes.

In one implementation, the second temperature is above about 25° C. In another implementation, the method further comprises lowering the second temperature to a third temperature. In one implementation, the water purification compound comprises sodium dichloroisocyanurate, chlorine, chloramines, chlorine dioxide, polyaluminium chloride, aluminum sulfate, ferric sulfate, hydrogen peroxide, sodium hydroxide bromide, silver nanoparticles, iron, iodine, activated carbon, or any combination thereof.

In one implementation, the shell comprises a water-soluble compound. In one implementation, the shell comprises one or more of polyvinyl alcohol, polyvinylpyrrolidone (PVP), carrageenan, gelatin, hydroxypropyl methylcellulose (HPMC), pullulan, starch film, benzoxaborole-poly(vinyl alcohol) (benzoxaborole-PVA), pectin, or any combination thereof.

In one implementation, the one or more reagent is a sequencing reagent, a sample preparation reagent, a library preparation reagent, or a combination thereof. In one implementation, the one or more reagent is selected from one or more enzyme, salt, surfactant, buffering agent, enzyme inhibitor, primer, nucleotide, organic osmolite, magnetic bead, molecular probe, crowding agent, small molecule, labelled-nucleotide, or any combination thereof.

In one implementation, the interior compartment comprises one or more dry reagent, one or more microsphere, one or more bead, one or more powder, one or more cake, one or more gel, one or more liquid, or any combination thereof. In one implementation, the one or more reagent is lyophilised. In one implementation, the interior compartment comprises a plurality of microspheres comprising a plurality of reagents. In another implementation, the interior compartment comprises a plurality of microspheres comprising one reagent.

A ninth aspect relates to a cartridge. The cartridge includes a reagent reservoir, wherein the reagent reservoir comprises a composition, said composition comprising: a shell surrounding an interior compartment, wherein said interior compartment comprises one or more reagent and wherein said shell releases said interior compartment when said shell is exposed to a first release condition, wherein said interior compartment releases said one or more reagent when said interior compartment is exposed to a second release condition, and wherein said first release condition is different from said second release condition.

In one implementation, the cartridge comprises a water purification compound. In one implementation, the water purification compound comprises sodium dichloroisocyanurate, chlorine, chloramines, chlorine dioxide, polyaluminium chloride, aluminum sulfate, ferric sulfate, hydrogen peroxide, sodium hydroxide bromide, silver nanoparticles, iron, iodine, activated carbon, or any combination thereof.

In one implementation, the first release condition is exposure to a liquid.

In one implementation, the second release condition is exposure to a temperature above about 25° C.

A tenth aspect relates to a cartridge. The cartridge comprises a reagent reservoir, wherein the reagent reservoir comprises a composition, said composition comprising: a dissolvable first shell, and a dissolvable second shell, the second shell comprising one or more reagent.

In one implementation, the cartridge comprises a water purification compound. In one implementation, the water purification compound comprises sodium dichloroisocyanurate, chlorine, chloramines, chlorine dioxide, polyaluminium chloride, aluminum sulfate, ferric sulfate, hydrogen peroxide, sodium hydroxide bromide, silver nanoparticles, iron, iodine, activated carbon, or any combination thereof.

In one implementation, the first release condition is exposure to a liquid. In one implementation, the second release condition is exposure to a temperature above about 25° C. In one implementation, the first shell is an exterior shell. In one implementation, the second shell is an interior shell.

An eleventh aspect relates to a system for controlling release of one or more reagent. The system includes a well; a composition comprising: a shell surrounding an interior compartment, wherein said interior compartment comprises one or more reagent, and wherein said shell releases said interior compartment when said shell is exposed to a first release condition, wherein said interior compartment releases said one or more reagent when said interior compartment is exposed to a second release condition, and wherein said first release condition is different from said second release condition; and a liquid.

In one implementation, the liquid is in the well. In another implementation, the composition is in the well. In another implementation, the system further comprises a temperature controller on the well.

In one implementation, the system further comprises a water purification compound. In one implementation, the water purification compound comprises sodium dichloroisocyanurate, chlorine, chloramines, chlorine dioxide, polyaluminium chloride, aluminum sulfate, ferric sulfate, hydrogen peroxide, sodium hydroxide bromide, silver nanoparticles, iron, iodine, activated carbon, or any combination thereof.

A twelfth aspect relates to a system for controlling release of one or more reagent. The system includes a well; a composition comprising: a dissolvable first shell, and a dissolvable second shell, the second shell comprising one or more reagent; and a liquid.

In one implementation, the liquid is in said well. In one implementation, the composition is in said well. In one implementation, the system further includes a temperature controller on said well.

In one implementation, the system further includes a water purification compound. In one implementation, the water purification compound comprises sodium dichloroisocyanurate, chlorine, chloramines, chlorine dioxide, polyaluminium chloride, aluminum sulfate, ferric sulfate, hydrogen peroxide, sodium hydroxide bromide, silver nanoparticles, iron, iodine, activated carbon, or any combination thereof. In one implementation, the first shell is an exterior shell. In one implementation, the second shell is an interior shell.

A thirteenth aspect relates to a method. The method includes: flowing a liquid having a temperature into a well, where the well comprises a capsule, where the capsule comprises a first shell surrounding a water purification compound and a second shell surrounding one or more reagent, wherein said first shell releases said water purification compound upon exposure to a first release condition, wherein said second shell releases said one or more reagent upon exposure to a second release condition, wherein said first release condition is different from said second release condition, wherein said water purification compound substantially or completely degrades upon exposure to a degradation condition; exposing said first shell to the first release condition whereby the water purification compound is released; exposing said water purification compound to the degradation condition whereby said water purification compound is substantially or completely degraded; and exposing said second shell condition to the second release condition whereby said one or more reagent is released.

In one implementation, the first release condition is exposure to the liquid. In another implementation, the degradation condition is an elevated temperature of the liquid. In one implementation, the elevated temperature is greater than or equal to about 25° C. In one implementation, the degradation condition is the same as the second release condition. In one implementation, flowing a liquid, exposing said first shell to the first release condition, and exposing said water purification compound to the degradation condition are performed in order. In another implementation, flowing a liquid, exposing said first shell to the first release condition, exposing said water purification compound to the degradation condition, and exposing said second shell condition to the second release condition are performed in order.

In accordance with the present disclosure, the compositions, methods, cartridges, and systems described herein have many advantages.

The problem of staggering the dissolution of multiple reagent capsules within a common well can be solved using a sequential release system as described herein. The problem of differentiating between different reagents in terms of dissolving time can be addressed through the combined use of different thicknesses of water-soluble film, different compositions of water-soluble film, and water-soluble films which use different release triggers in order to undergo reagent release as described herein. The problem of purifying atmospheric-captured water can be overcome using water purification compounds, such as sodium dichloroisocyanurate, and such water purification compounds may be integrated into a reagent capsule composition, method, cartridge, and system as described herein.

### BRIEF DESCRIPTION OF THE DRAWINGS

FIG. 1 shows a water purification compound in sequential workflow. A water purification compound may be placed in an atmospheric-captured water tank as a grand water purification tablet or as a small tablet incorporated into reagent capsules and of a size proportionate to the amount of liquid that the capsule will rehydrate to.

FIG. 2 shows the manufacture of a reagent capsule with a water purification compound incorporated therein.

FIG. 3 shows that the size of the water purification compound scales with the rehydration volume or the volume of the final reagent mix. A whole reagent capsule is shown on the left and a reagent component capsule is shown on the right.

FIG. 4 shows a workflow for encapsulated reagent microspheres with a water purifying compound.

FIG. 5 shows a composition design having a common well configuration. A water purification compound may be placed in a tank, or with a composition, tablet, or capsule.

FIG. 6 shows one implementation of the compositions described herein with a shell surrounding an interior compartment.

FIG. 7 shows one implementation of the compositions described herein with three separate compositions: a first shell surrounding an interior compartment, a second shell surrounding a second interior compartment, and a third shell surrounding a third interior compartment.

FIG. 8 shows a composition as described herein under one or more release conditions as described herein.

FIG. 9 shows one implementation of the one or more reagent in the compositions described herein, in particular, a lyophilised microsphere.

FIG. 10 depicts one implementation of the one or more reagent in the compositions described herein, in particular, a lyophilised microsphere.

FIG. 11 is a flow chart describing one aspect described herein for a method for controlling release of one or more reagent.

FIG. 12 is a flow chart describing one aspect described herein for a method for controlling release of one or more reagent.

FIG. 13 is a flow chart describing one aspect described herein for a method for controlling release of one or more reagent.

FIG. 14 is a flow chart describing one aspect described herein for a method.

FIG. 15 is a flow chart describing one aspect described herein for a method.

FIG. 16 is a flow chart describing one aspect described herein for a method.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits and advantages described herein.

### DETAILED DESCRIPTION

A first aspect relates to a composition. The composition includes a shell surrounding an interior compartment, wherein said interior compartment comprises one or more reagent and wherein said shell releases said interior compartment when said shell is exposed to a first release condition, wherein said interior compartment releases said one or more reagent when said interior compartment is exposed to a second release condition, and wherein said first release condition is different from the second release condition.

It is to be appreciated that certain aspects, modes, implementations, variations, and features of the present disclosure are described below in various levels of detail in order to provide a substantial understanding of the present technology. Unless otherwise noted, all technical and scientific terms used herein generally have the same meaning as commonly understood by one of ordinary skill in the art. The use of the term “including” as well as other forms is not limiting. The use of the term “having” as well as other forms is not limiting. As used in this disclosure, whether in a transitional phrase or in the body of the claim, the terms “comprise(s)” and “comprising” are to be interpreted as having an open-ended meaning. That is, the terms are to be interpreted synonymously with the phrases “having at least” or “including at least.”

The terms “substantially”, “approximately”, “about”, “relatively”, or other such similar terms that may be used throughout this disclosure, including the claims, are used to describe and account for small fluctuations, such as due to variations in processing, from a reference or parameter. Such small fluctuations include a zero fluctuation from the reference or parameter as well. For example, fluctuations can refer to less than or equal to ±10%, such as less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

It is further appreciated that certain features described herein, which are, for clarity, described in the context of separate implementations, can also be provided in combination in a single implementation. Conversely, various features which are, for brevity, described in the context of a single implementation, can also be provided separately or in any suitable sub-combination.

The terms “connect”, “contact”, and/or “coupled” include a variety of arrangements and assemblies. These arrangements and techniques include, but are not limited to, (1) the direct joining of one component and another component with no intervening components therebetween (i.e., the components are in direct physical contact); and (2) the joining of one component and another component with one or more components therebetween, provided that the one component being “connected to” or “contacting” or “coupled to” the other component is somehow in operative communication (e.g., electrically, fluidly, physically, optically, etc.) with the other component (optionally with the presence of one or more additional components therebetween). Components that are in direct physical contact with one another may or may not be in electrical contact and/or fluid contact with one another. Moreover, two components that are electrically connected, electrically coupled, optically connected, optically coupled, fluidly connected, or fluidly coupled may or may not be in direct physical contact, and one or more other components may be positioned between those two connected components.

As described herein, the term “attached” may include when two things are joined, fastened, adhered, connected, or bound to one another. A reaction component, like a polymerase, can be attached to a solid phase component, like a conductive channel, by a covalent or a non-covalent bond. As described herein, the phrase “covalently attached” or “covalently bonded” refers to forming one or more chemical bonds that are characterized by the sharing of pairs of electrons between atoms. A non-covalent bond is one that does not involve the sharing of pairs of electrons and may include, for example, hydrogen bonds, ionic bonds, van der Waals forces, hydrophilic interactions, and hydrophobic interactions.

As described herein, the terms “polynucleotide” or “nucleic acids” refer to deoxyribonucleic acid (DNA), ribonucleic acid (RNA), or analogs of either DNA or RNA made from nucleotide analogs. The terms as used herein also encompasses cDNA, that is complementary, or copy DNA produced from an RNA template, for example by the action of reverse transcriptase. In one implementation, the nucleic acid to be analyzed, for example by sequencing through use of the described systems, is immobilized on a substrate (e.g., a substrate within a flow cell or one or more beads upon a substrate such as a flow cell, etc.). The term immobilized as used herein is intended to encompass direct or indirect, covalent, or non-covalent attachment, unless indicated otherwise, either explicitly or by context. The analytes (e.g., nucleic acids) may remain immobilized or attached to the support under conditions in which it is intended to use the support, such as in applications requiring nucleic acid sequencing. In one implementation, the template polynucleotide is one of a plurality of template polynucleotides attached to a substrate. In one implementation, the plurality of template polynucleotides attached to the substrate include a cluster of copies of a library polynucleotide as described herein.

Nucleic acids include naturally occurring nucleic acids or functional analogs thereof. Particularly useful functional analogs are capable of hybridizing to a nucleic acid in a sequence specific fashion or capable of being used as a template for replication of a particular nucleotide sequence. Naturally occurring nucleic acids generally have a backbone containing phosphodiester bonds. An analog structure can have an alternate backbone linkage including any of a variety of those known in the art such as peptide nucleic acid (PNA) or locked nucleic acid (LNA). Naturally occurring nucleic acids generally have a deoxyribose sugar (e.g., found in deoxyribonucleic acid (DNA)) or a ribose sugar (e.g., found in ribonucleic acid (RNA)).

In RNA, the sugar is a ribose, and in DNA a deoxyribose, i.e., a sugar lacking a hydroxyl group that is present in ribose. The nitrogen containing heterocyclic base can be purine or pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose may be bonded to N-1 of a pyrimidine or N-9 of a purine.

A nucleic acid can contain any of a variety of analogs of these sugar moieties that are known in the art. A nucleic acid can include native or non-native bases. A native deoxyribonucleic acid can have one or more bases selected from the group consisting of adenine, thymine, cytosine, or guanine and a ribonucleic acid can have one or more bases selected from the group consisting of uracil, adenine, cytosine or guanine. Useful non-native bases that can be included in a nucleic acid are known in the art.

The term nucleotide as described herein may include natural nucleotides, analogs thereof, ribonucleotides, deoxyribonucleotides, dideoxyribonucleotides and other molecules known as nucleotides. As described herein, a nucleotide may include a nitrogen containing heterocyclic base, a sugar, and one or more phosphate groups. Nucleotides may be monomeric units of a nucleic acid sequence, for example to identify a subunit present in a DNA or RNA strand. A nucleotide may also include a molecule that is not necessarily present in a polymer, for example, a molecule that is capable of being incorporated into a polynucleotide in a template dependent manner by a polymerase. A nucleotide may include a nucleoside unit having, for example, 0, 1, 2, 3 or more phosphates on the 5′ carbon. Tetraphosphate nucleotides, pentaphosphate nucleotides, and hexaphosphate nucleotides may be useful, as may be nucleotides with more than 6 phosphates, such as 7, 8, 9, 10, or more phosphates, on the 5′ carbon. Examples of naturally occurring nucleotides include, without limitation, ATP, UTP, CTP, GTP, ADP, UDP, CDP, GDP, AMP, UMP, CMP, GMP, dATP, dTTP, dCTP, dGTP, dADP, dTDP, dCDP, dGDP, dAMP, dTMP, dCMP, and dGMP.

Non-natural nucleotides include nucleotide analogs, such as those that are not present in a natural biological system or not substantially incorporated into polynucleotides by a polymerase in its natural milieu, for example, in a non-recombinant cell that expresses the polymerase. Non-natural nucleotides include those that are incorporated into a polynucleotide strand by a polymerase at a rate that is substantially faster or slower than the rate at which another nucleotide, such as a natural nucleotide that base-pairs with the same Watson-Crick complementary base, is incorporated into the strand by the polymerase. For example, a non-natural nucleotide may be incorporated at a rate that is at least 2 fold different, 5 fold different, 10 fold different, 25 fold different, 50 fold different, 100 fold different, 1000 fold different, 10000 fold different, or more when compared to the incorporation rate of a natural nucleotide. A non-natural nucleotide can be capable of being further extended after being incorporated into a polynucleotide. Examples include, nucleotide analogs having a 3′ hydroxyl or nucleotide analogs having a reversible terminator moiety at the 3′ position that can be removed to allow further extension of a polynucleotide that has incorporated the nucleotide analog. Examples of reversible terminator moieties are described, for example, in U.S. Pat. No. 7,427,673, which is hereby incorporated by reference in its entirety. It will be understood that in some implementations a nucleotide analog having a 3′ terminator moiety or lacking a 3′ hydroxyl (such as a dideoxynucleotide analog) can be used under conditions where the polynucleotide that has incorporated the nucleotide analog is not further extended. In some implementations, nucleotide(s) may not include a reversible terminator moiety, or the nucleotides(s) will not include a non-reversible terminator moiety or the nucleotide(s) will not include any terminator moiety at all. In one implementation, the 3′-hydroxy blocking group is a reversible blocking group.

The term “cluster” refers to a discrete site on a solid support comprised of a plurality of identical immobilized nucleic acid strands and a plurality of identical immobilized complementary nucleic acid strands. The term “clustered array” refers to an array formed from such clusters or colonies. In this context, the term “array” is not to be understood as requiring an ordered arrangement of clusters.

As used herein, the term “different,” when used in reference to nucleic acids, means that the nucleic acids have nucleotide sequences that are not the same as each other. Two or more nucleic acids can have nucleotide sequences that are different along their entire length. Alternatively, two or more nucleic acids can have nucleotide sequences that are different along a substantial portion of their length. For example, two or more nucleic acids can have target nucleotide sequence portions that are different from each other while also having a universal sequence region that are the same as each other.

As used herein, a “library” is a population of polynucleotides from a given source or sample. A library comprises a plurality of target polynucleotides.

A modified nucleotide as described herein includes one that has a purine or pyrimidine base and a sugar moiety having a 3′-hydroxy blocking group. In one implementation, the modified nucleotide is linked to a detectable label. In one implementation, the detectable label comprises a fluorophore. This disclosure encompasses nucleotides including a fluorescent label (or any other detection tag) that may be used in any method disclosed herein, on its own or incorporated into or associated with a larger molecular structure or conjugate. Additional examples of detectable labels are described in U.S. Pat. No. 7,541,444, which is hereby incorporated by reference in its entirety.

The fluorescent label can include compounds selected from any known fluorescent species, for example rhodamines or cyanines. A fluorescent label as disclosed herein may be attached to any position on a nucleotide base, and may optionally include a linker. In one implementation, the modified nucleotide is linked to a detectable label via a cleavable linker. The function of the linker is generally to aid chemical attachment of the fluorescent label to the nucleotide. In particular implementations, Watson-Crick base pairing can still be carried out for the resulting analogue. A linker group may be used to covalently attach a dye to the nucleoside or nucleotide. A linker moiety may be of sufficient length to connect a nucleotide to a compound such that the compound does not significantly interfere with the overall binding and recognition of the nucleotide by a nucleic acid replication enzyme. Thus, the linker can also include a spacer unit. The spacer distances, for example, the nucleotide base from a cleavage site or label.

The linker may be cleavable and the cleavage site may be located at a position on the linker that results in part of the linker remaining attached to the nucleotide base after cleavage or that results in the whole linker being removed from the nucleotide base. Exemplary linkers include azide- and allyl-containing cleavable moieties, disulfide linkers, acid labile moieties (including dialkoxybenzyl moieties, Sieber linkers, indole moieties, t-butyl Sieber moieties), electrophilically cleavable moieties, nucleophilically cleavable moieties, photocleavable moieties, cleavage under reductive conditions, oxidative conditions, cleavage via use of safety-catch moieties, and cleavage by elimination mechanisms. Examples of such moieties are described in WO03/048387, which is hereby incorporated by reference in its entirety.

The composition may include different modified nucleotides linked to different detectable labels. In some implementations, four different modified nucleotides may be linked to four different detectable labels. Alternatively, four different modified nucleotides may be labeled with two different detectable labels (for example, for two-channel sequencing by synthesis) or with a single detectable label (for example, for one-channel sequencing by synthesis).

As used herein, a “nucleoside” is structurally similar to a nucleotide, but is missing the phosphate moieties. An example of a nucleoside analogue is one in which the label is linked to the base and there is no phosphate group attached to the sugar molecule. The term “nucleoside” is used herein in its ordinary sense as understood by those skilled in the art. Examples include, but are not limited to, a ribonucleoside including a ribose moiety and a deoxyribonucleoside including a deoxyribose moiety. A modified pentose moiety is a pentose moiety in which an oxygen atom is replaced with a carbon and/or a carbon is replaced with a sulfur or an oxygen atom. A “nucleoside” is a monomer that may have a substituted base and/or sugar moiety.

The term “purine base” is used herein in its ordinary sense as understood by those skilled in the art, and includes its tautomers. Similarly, the term “pyrimidine base” is used herein in its ordinary sense as understood by those skilled in the art, and includes its tautomers. A non-limiting list of optionally substituted purine-bases includes purine, adenine, guanine, hypoxanthine, xanthine, alloxanthine, 7-alkylguanine (e.g. 7-methylguanine), theobromine, caffeine, uric acid and isoguanine. Examples of pyrimidine bases include, but are not limited to, cytosine, thymine, uracil, 5,6-dihydrouracil and 5-alkylcytosine (e.g., 5-methylcytosine).

The term substrate (or solid support), as described herein, may include any inert substrate or matrix to which nucleic acids can be attached, such as for example glass surfaces, plastic surfaces, latex, dextran, polystyrene surfaces, polypropylene surfaces, polyacrylamide gels, gold surfaces, and silicon wafers. For example, a substrate may be a glass surface (e.g., a planar surface of a flow cell channel). In one implementation, a substrate may include an inert substrate or matrix which is “functionalized,” such as by applying a layer or coating of an intermediate material including reactive groups which permit covalent attachment to molecules such as polynucleotides. Supports may include polyacrylamide hydrogel supported on an inert substrate such as glass. Molecules (e.g., polynucleotides) may be directly covalently attached to an intermediate material (e.g., a hydrogel). A support may include a plurality of particles or beads each having a different attached analyte.

As used herein, “derivative” or “analogue” means a synthetic nucleotide or nucleoside derivative having modified base moieties and/or modified sugar moieties. Such derivatives and analogs are discussed in, for example, Bücher, N. “Nucleotide Analogs. Synthesis and Biological Function,” Angewandte Chemie 97:564 (1980), which is hereby incorporated by reference in its entirety. Nucleotide analogs may also include modified phosphodiester linkages, including phosphorothioate, phosphorodithioate, alkyl-phosphonate, phosphoranilidate and phosphoramidate linkages. “Derivative”, “analog”, and “modified” as used herein, may be used interchangeably, and are encompassed by the terms “nucleotide” and “nucleoside” as described herein.

As used herein, the terms “solid phase” or “surface” are used to mean either a planar array wherein primers are attached to a flat surface, for example, glass, silica or plastic microscope slides or similar flow cell devices; beads, wherein either one or two primers are attached to the beads and the beads are amplified; or an array of beads on a surface after the beads have been amplified.

As used herein, “substantially free of” a material (including, for example, a crowding agent or a nucleic acid) refers to compositions having less than 10% of the material, less than 5% of the material, less than 4% of the material, less than 3% of the material, less than 2% of the material, or less than 1% of the material.

As described herein, a “shell” includes a composition that surrounds an interior compartment. The interior compartment as described herein includes one or more reagent. As described herein, the shell in the composition releases the interior compartment when the shell is exposed to a first release condition. The interior compartment of the composition described herein releases one or more reagent when the interior compartment is exposed to a second release condition. The interior compartment may, for example, have its own interior compartment shell that surrounds the one or more reagent. The first release condition may be different from the second release condition.

In one implementation, the shell includes a water-soluble compound. In one implementation, the shell includes, for example, a material selected from one or more of polyvinyl alcohol, polyvinylpyrrolidone (PVP), carrageenan, gelatin, hydroxypropyl methylcellulose (HPMC), pullulan, starch film, benzoxaborole-poly(vinyl alcohol) (benzoxaborole-PVA), pectin, or any combination thereof. In one implementation, the shell may include one or more of a polymethacrylate-based copolymers, acrylic polymers, water-soluble polymers, poly(N-isopropylacrylamide), pluronic, wax, azobenzene, photochromic markers such as spirobenzopyran, gold nanoparticles, polyvinyl alcohol (PVA), poly(lactic-co-glycolic acid) (PLGA), alginate, gellan, poly(disulfide), metal organic frameworks (MOF), and any combination thereof. Examples of useful copolymers include those derived from esters of acrylic and methacrylic acids, polyvinyl alcohol-polyethylene glycol graft copolymers, and combinations of polyvinyl acetate phthalate (Phthalavin enteric coating polymer) and plasticizers.

The amount of shell material includes, for example, any amount suitable to produce a desired shell result. In one implementation, the shell material is present in an amount between about 1 wt % and about 100 wt % of the shell. For example, the shell material may be present in about 1 wt %, 2 wt %, 3 wt %, 4 wt %, 5 wt %, 6 wt %, 7 wt %, 8 wt %, 9 wt %, 10 wt %, 15 wt %, 20 wt %, 25 wt %, 30 wt %, 35 wt %, 40 wt %, 45 wt %, 50 wt %, 60 wt %, 70 wt %, 80 wt %, 90 wt %, 100 wt %, of the shell, or any amount therebetween. In one implementation, the shell material is present in an amount between about 10 wt % and about 90 wt %, or between about 10 wt % and about 80 wt %, or between about 10 wt % and about 70 wt %, or between about 10 wt % and about 60 wt %, or between about 10 wt % and about 50 wt %, of the shell.

As described herein, “encapsulate”, “encapsulated”, and “encapsulation” include the enclosing of one or more compositions as described herein. Microencapsulation as described herein refers to the embedding of at least one ingredient, for example, an active agent, into at least one other material, for example, a shell material. Encapsulation in accordance with the present disclosure includes, but is not limited to, bulk encapsulation, matrix encapsulation, macroencapsulation, microencapsulation, nano encapsulation, single molecule, and ionic encapsulation.

In accordance with the present disclosure, the compositions, methods, cartridges, and systems described herein have many advantages and benefits including, for example, increasing stability of reagents, use of macroencapsulation to enable multi-run cartridges, and use of microencapsulation to enable simplified workflows and reduced number of reagent wells. The compositions, methods, cartridges, and systems described herein use encapsulation of particles that would otherwise be responsive to pH changes to stabilize these buffers and increase SBS performance. The compositions, methods, cartridges, and systems described herein also use encapsulation to reduce the risk of static charge that otherwise presents difficulty for dispensing and dry compounding reagents during manufacturing. High static charge further presents difficulty in predicting distribution of lyophilised content in a well. For example, a high static charge may result in well contents failing to settle to the bottom of a well which contributes to difficulty in obtaining desired rehydration of the lyophilised content. A shell as described herein may include, for example, biodegradable polymers.

The first release condition as described herein may, in one implementation, include a temperature-controlled release condition, a pH-controlled release condition, a time-controlled release condition, a position-controlled release condition, or any combination thereof. The first release condition may be based on a particular temperature, pH, period of time, or position that is suitable to dissolve the shell or release the interior compartment.

The second release condition as described herein may, in one implementation, include a temperature-controlled release condition, a pH-controlled release condition, a time-controlled release condition, a position-controlled release condition, or any combination thereof. The second release condition may be based on a particular temperature, pH, period of time, or position that is suitable to dissolve the interior compartment or release the one or more reagent. The first release condition and second release condition are separate and independent of one another.

“Modifying” any of the conditions as described herein (e.g., the first release condition or the second release condition) includes any change in one or more conditions in the composition and, or in the alternative, the environment surrounding the composition (e.g., a rehydration solution or other surrounding solution). Modifying the conditions in one implementation allows for a sequential release of any compound in the composition or release of one or more reagents in the interior compartment. One way to enable sequential release of reagents is through temperature-triggered release. Other reaction characteristics may be modified in addition to or instead of time and, or in the alternative, temperature. For example, pH and humidity may be modified to further control release of one or more compounds, components, and reagent(s) contained therein. The conditions may be modified any number of times to produce any number of different conditions.

In one implementation, an additional composition is provided, and mixed under a third condition effective to control release of one or more reagent from the additional composition. In one implementation, reagent components are segregated, and may thereby prevent and control undesired interactions. The third or other subsequent condition as described herein may, in one implementation, include a temperature-controlled release condition, a pH-controlled release condition, a time-controlled release condition, a position-controlled release condition, or any combination thereof. The third or other subsequent release condition may be based on a particular temperature, pH, period of time, or position that is suitable to dissolve the interior compartment or release the one or more reagent therein. The third release condition is separate and independent of the first release condition and the second release condition.

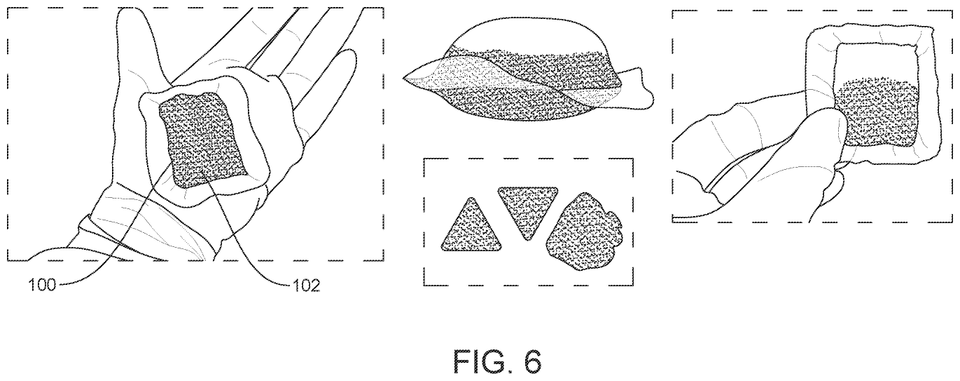

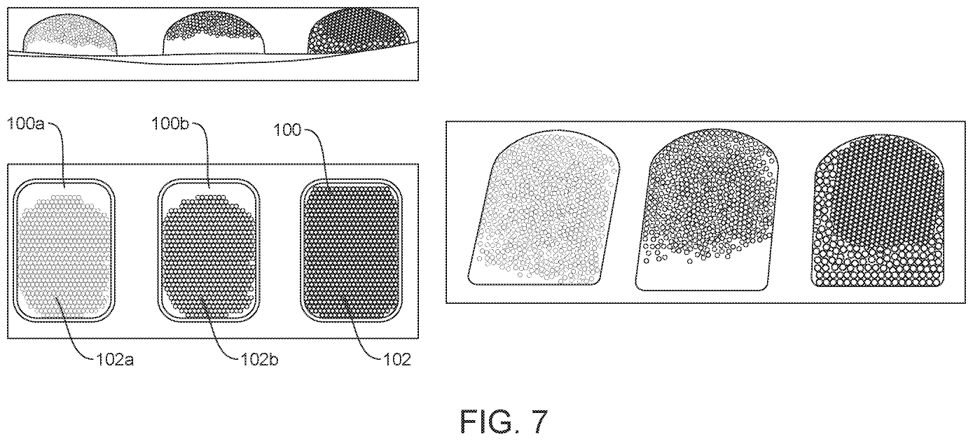

In one implementation, as shown for example in FIGS. 6 and 7, the composition includes a shell 100 (e.g., 100a, 100b, 100c, etc.) that surrounds an interior compartment 102 (e.g., 102a, 102b, 102c, etc.). Shell 100 may be referred to herein as a first shell or a dissolvable first shell or an exterior shell. Interior compartment 102 includes at least one reagent. Interior compartment 102 may also or alternatively include one or more water purification compounds. Interior compartment 102 may be referred to herein as a second shell or a dissolvable second shell and may include an interior shell. Shell 100 may, for example, release interior compartment 102 when shell 100 is exposed to a first release condition. Interior compartment 102 may, for example, release one or more reagent that is positioned inside interior compartment 102. The composition may include a plurality of compositions, or may be used in conjunction with one or more additional compositions that include a shell 100 (e.g., 100a, 100b, 100c, etc.) and an interior compartment 102 (e.g., 102a, 102b, 102c) as shown in FIG. 7, that may include different reagents, the same reagents, or substantially the same reagents. Furthermore, shell 100 (e.g., 100a, 100b, 100c, etc.) and interior compartment 102 (e.g., 102a, 102b, 102c) may respond to different release conditions, the same release conditions, or substantially similar release conditions. The release conditions may be in accordance with those described herein.

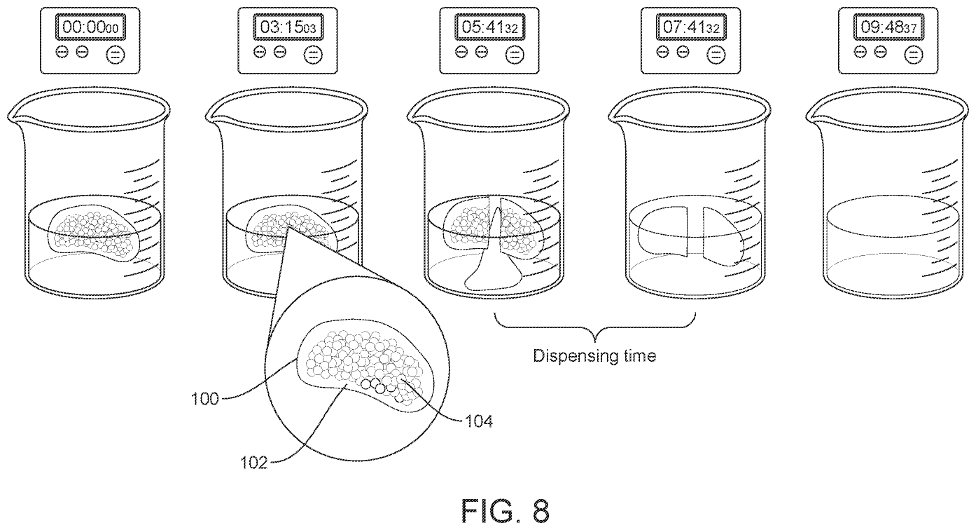

In one example, as shown in FIG. 8, shell 100 surrounds interior compartment 102 and interior compartment 102 includes a plurality of reagents 104. The plurality of reagents may be the same type of reagent or different types of reagents and may be dry or substantially dry (e.g., lyophilised) as shown in FIGS. 8-10. The composition, when placed in a first release condition, may release interior compartment 102. The composition, when placed in a second release condition, may release one or more reagent 104. In one implementation, the first release condition may release a first reagent 104a into a surrounding liquid environment. In another implementation, optionally after first reagent 104a is released, the second release condition may release a second reagent 104b.

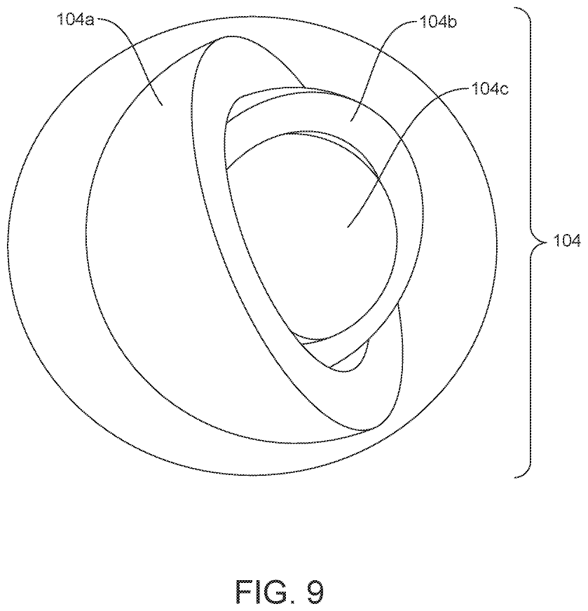

As shown in FIG. 9, reagent 104 may be located in, or formed as, for example, a microsphere. Reagent 104 may contain a plurality of reagents that may be the same type of reagent or different types of reagents. FIG. 9 shows one implementation where reagent 104 includes three reagents: reagent 104a is located in an outer layer or shell of reagent 104 (also referred to herein as an interior shell); reagent 104b is located in a middle layer or shell of reagent 104; reagent 104c is located in a core of reagent 104. Any of reagents 104a, 104b, and/or 104c, along with any additional reagents may be organized in concentric circles in a microsphere, or may alternatively be adjacent to one another.

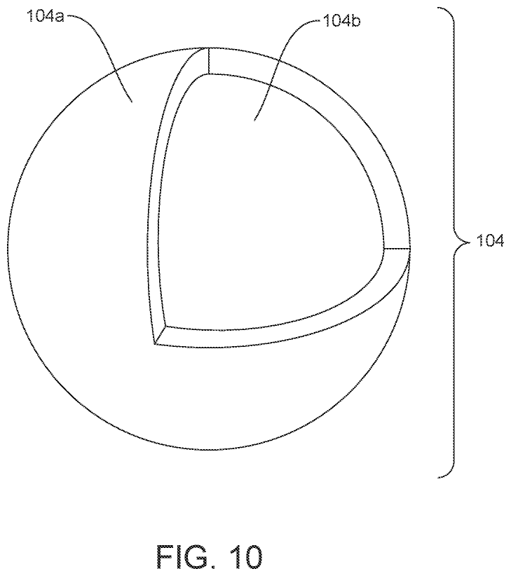

FIG. 10 shows one implementation of reagent 104, where reagent 104 is a microencapsulated lyophilised microsphere. In such an example, reagent 104 may contain a single type of reagent or a plurality of reagent types. An encapsulated lyophilised microsphere as described herein may include one, two, three, or more than three types of reagent. An encapsulated lyophilised microsphere as described herein may contain an outer layer or shell 104a (also referred to herein as an interior shell) and a core (e.g., 104b or 104c) as shown in FIGS. 9 and 10, and each of the shell and core may optionally contain one or more reagents that are the same or different. In one implementation, the core of a microsphere contains a reagent (as shown, for example, as 104b in FIG. 10) and may be located inside the microsphere shell which may contain the same or different reagent as the core (as shown, for example, as 104a in FIG. 10). The lyophilised microsphere may further include a third reagent or any number of additional reagents to make the microsphere useful for applications described herein. In one implementation, the core of a microsphere contains a reagent (as shown, for example, as 104c in FIG. 9) and may be located inside a middle layer of the microsphere (as shown, for example, as 104b in FIG. 9), which are both located inside an outer layer or shell of the microsphere (as shown, for example, as 104c in FIG. 9). Each of the reagents may be different. In one implementation, a first reagent may be different from a second reagent. In one implementation, a first reagent may be different from a third reagent. In other implementations, a second reagent may be different from a third reagent. Alternatively, the reagents in the lyophilised microsphere may be the same or substantially similar. For example, a first reagent may be the same or substantially similar to a third reagent. A first reagent may likewise be the same or substantially similar to a second reagent. A second reagent may be the same or substantially similar to a third reagent. In certain implementations, the diameter of the reagent 104b is between 470 μm and 500 μm, for example, about 484 μm. In certain implementations, the thickness of the shell is between 4 μm and 5 μm, for example, about 4.6 μm.

Each of the reagents in the compositions described herein may respond to different release conditions. In certain implementations, a first, second, and/or third reagent may respond to different release conditions. In certain implementations, a first and third reagent respond to different release conditions. In certain implementations, a first and second reagent respond to different release conditions. In other implementations, a second and third reagent respond to different release conditions. Alternatively, a first, second, and/or third reagent may respond to the same or substantially similar release conditions. In certain implementations, a first and third reagent respond to the same or substantially similar release conditions. In certain implementations, a first and second reagent respond to the same or substantially similar release conditions. In certain implementations, a second and third reagent respond to the same or substantially similar release conditions.

In one implementation, the shell releases the interior compartment when the shell is exposed to at least one additional shell release condition, where one or more of the at least one additional shell release condition is different from the first release condition. In one implementation, the interior compartment prevents release of the one or more reagent when the shell is exposed to the at least one additional shell release condition. In another implementation, the interior compartment releases the one or more reagent when the interior compartment is exposed to at least one additional interior compartment release condition, where one or more of the at least one additional interior compartment release condition is different from the second release condition. The additional shell release condition and the interior compartment release condition may be in addition, or in the alternative, to the first and second release condition.

The compositions, methods, cartridges, and systems described herein provide for timed-release so that various components and reagents may be released at different times, for example, in a sequential or otherwise controlled manner. The rate of release may be adjustable to allow for controlled-release of composition components and reagents. The rate of release may be for any suitable period of time. For example, a shell or an interior compartment may release or dissolve over a short-period of time such as 1 minute or less (e.g., less than 1 second, 1 second, 10 seconds, 20 seconds, 30 seconds, 45 seconds, 60 seconds, or any period of time therebetween). Alternatively, a shell or an interior compartment may release or dissolve over an intermediate-period of time such as between 1 minute and 30 minutes (e.g., 1 minute, 5 minutes, 10 minutes, 15 minutes, 20 minutes, 25 minutes, 30 minutes, or any period of time therebetween). Alternatively, a shell or an interior compartment may release or dissolve over a long-period of time such as more than 30 minutes (e.g., 35 minutes, 40 minutes, 45 minutes, 50 minutes, 55 minutes, 60 minutes, 75 minutes, 90 minutes, 105 minutes, 120 minutes, more than 120 minutes, or any period of time therebetween). For example, a shell or an interior compartment as described herein may release quickly (e.g., in under one minute) at a low pH (e.g., between about 2-6), while that same shell may release slowly (e.g., in about 30 or more minutes) at a high pH (e.g., between about 10-14). Likewise, a shell or an interior compartment as described herein may release slowly (e.g., in about 30 or more minutes) at a low pH (e.g., between about 2-6), while that same shell may release quickly (e.g., in under one minute) at a high pH (e.g., between about 10-14). Similarly, a shell or interior compartment as described herein may release quickly (e.g., in under one minute) at an elevated temperature (e.g., above about 25° C.), while that same shell or interior compartment may release slowly (e.g., in about 30 or more minutes) at a lower temperature (e.g., at or below about 25° C.). In another example, a shell or interior compartment as described herein may release slowly (e.g., in about 30 or more minutes) at an elevated temperature (e.g., above about 25° C.), while that same shell or interior compartment may release quickly (e.g., in under one minute) at a lower temperature (e.g., at or below about 25° C.). In one implementation, either or both of the first and second conditions comprise a change in temperature. The temperature, for example, may be elevated to a temperature above about 25° C. Alternatively, the temperature, for example, may be reduced to at or below about 25° C.

In one implementation, the interior compartment prevents release of one or more reagent when the shell is exposed to the first release condition. As described herein, the second release condition (i.e., wherein the interior compartment releases one or more reagent) may be the same as the first release condition (i.e., wherein the shell releases the interior compartment). The shell and interior compartment may, in one implementation, release under the same condition. In such an instance, the shell and interior compartment may release or dissolve at different times. Alternatively, the shell and interior compartment may, in one implementation, release under the different conditions. In one implementation, the first release condition occurs before the second release condition. In another implementation, the second release condition occurs after the first release condition.

As described herein, preventing release of the one or more reagent when the shell is exposed to a first condition includes a prevention of release for at least an order of magnitude longer than under a second release condition. As described herein, preventing release of one or more reagent includes both complete prevention of reagent release and substantial delays in reagent release (i.e., preventing release of one or more reagent includes practically preventing release).

In one implementation, the shell has a shell width and the interior compartment has an interior compartment width, and the shell width is different from the interior compartment width. In one implementation, the shell width is between about 1 micrometer and about 1,000 micrometers. In another implementation, the interior compartment width is between about 1 micrometer and about 1,000 micrometers. The shell width may be, for example, about 1 micrometer, about 10 micrometers, about 25 micrometers, about 50 micrometers, about 75 micrometers, about 100 micrometers, about 125 micrometers, about 150 micrometers, about 175 micrometers, about 200 micrometers, about 225 micrometers, about 250 micrometers, about 275 micrometers, about 300 micrometers, about 325 micrometers, about 350 micrometers, about 375 micrometers, about 400 micrometers, about 450 micrometers, about 500 micrometers, about 550 micrometers, about 600 micrometers, about 650 micrometers, about 700 micrometers, about 750 micrometers, about 800 micrometers, about 850 micrometers, about 900 micrometers, about 950 micrometers, about 1,000 micrometers, or any amount therebetween. In one implementation, the shell width is between about 100 micrometers and 1,000 micrometers. In one implementation, one or both of the shell width or the interior compartment width is above 1,000 micrometers. In one implementation, the shell width is the same as the interior compartment width. As described herein, an “interior compartment” (interchangeably referred to as a “core” or “core region”) includes any material within the surrounding shell. An interior compartment in accordance with the present disclosure includes one or more reagents.

As used herein, the term “reagent” describes a single agent or a mixture of two or more agents useful for reacting with, interacting with, diluting, or adding to a sample, and may include the compositions described herein as well as agents used in nucleic acid reactions, including, for example buffers, chemicals, enzymes, polymerase, primers including those having a size of less than 50 base pairs, template nucleic acids, nucleotides, labels, dyes, or nucleases.

In one implementation, the one or more reagent is a sequencing reagent, a sample preparation reagent, a library preparation reagent, or a combination thereof. In one implementation, the one or more reagent is selected from one or more enzyme, salt, surfactant, buffering agent, enzyme inhibitor, primer, nucleotide, organic osmolite, magnetic bead, molecular probe, crowding agent, small molecule, labelled-nucleotide, or any combination thereof. In some implementations, the reagent may further or alternatively include a lysozyme, proteinase K, random hexamers, transposase (for example, Tn5), primers (for example, P5 and P7 adaptor sequences), ligase, catalyzing enzyme, deoxynucleotide triphosphates, buffers, or divalent cations. The reagent may further or alternatively include, for example, bead-linked transposomes (BLT), Tris pH7, MgCl₂, Mg acetate, Mg sulfate, indexed primers, Q5 polymerase, Bst3.0, Tris pH9, dNTPs, NaCl, betaine, or any combination thereof. A reagent as described herein may, in certain implementations, include enzymes such as polymerases, ligases, recombinases, or transposases; binding partners such as antibodies, epitopes, streptavidin, avidin, biotin, lectins or carbohydrates; or other biochemically active molecules. Other examples reagents include reagents for a biochemical protocol, such as a nucleic acid amplification protocol, an affinity-based assay protocol, an enzymatic assay protocol, a sequencing protocol, and/or a protocol for analyses of biological fluids. According to some implementations disclosed herein, a reagent may include one or more beads, in particular magnetic beads, depending on specific workflows and/or downstream applications.

In one implementation, a reagent in accordance with the present disclosure is a polymerase. As used herein, the term “polymerase” is intended to be consistent with its use in the art and includes, for example, an enzyme that produces a complementary replicate of a nucleic acid molecule using the nucleic acid as a template strand. Typically, DNA polymerases bind to the template strand and then move down the template strand sequentially adding nucleotides to the free hydroxyl group at the 3′ end of a growing strand of nucleic acid. DNA polymerases typically synthesize complementary DNA molecules from DNA templates and RNA polymerases typically synthesize RNA molecules from DNA templates (transcription). Polymerases can use a short RNA or DNA strand, called a primer, to begin strand growth. Some polymerases can displace the strand upstream of the site where they are adding bases to a chain. Such polymerases are said to be strand displacing, meaning they have an activity that removes a complementary strand from a template strand being read by the polymerase. Exemplary polymerases having strand displacing activity include, without limitation, the large fragment of Bst (Bacillus stearothermophilus) polymerase, exo-Klenow polymerase or sequencing grade T7 exo-polymerase. Some polymerases may degrade the strand in front of them, effectively replacing it with the growing chain behind (5′ exonuclease activity). Some polymerases have an activity that may degrade the strand behind them (3′ exonuclease activity). Some useful polymerases have been modified, either by mutation or otherwise, to reduce or eliminate 3′ and/or 5′ exonuclease activity.

Polymerase in accordance with the present disclosure may include any polymerase that can tolerate incorporation of a phosphate-labeled nucleotide. Examples of polymerases that may be useful in accordance with the present disclosure include but are not limited to phi29 polymerase, a klenow fragment, DNA polymerase I, DNA polymerase III, GA-1, PZA, phi15, Nf, G1, PZE, PRD1, B103, GA-1, 9oN polymerase, Bst, Bsu, T4, T5, T7, Taq, Vent, RT, pol beta, pol gamma, and combinations thereof. Polymerases engineered to have specific properties may be used. In one implementation, the polymerase may be useful for sequencing (“sequencing polymerase”). In one implementation, the reagent includes a polymerase, for example, Pol 812, 129 DNA polymerase, Taq polymerase, Bsu polymerase, or any combination thereof.

A primer as disclosed herein includes a nucleic acid molecule that can hybridize to a target sequence of interest. In several implementations, a primer may function as a substrate onto which nucleotides can be polymerized by a polymerase. However, in some examples, the primer can become incorporated into the synthesized nucleic acid strand and provide a site to which another primer can hybridize to prime synthesis of a new strand that is complementary to the synthesized nucleic acid molecule. The primer can include any combination of nucleotides or analogs thereof. In one implementation, the primer is a single-stranded oligonucleotide or polynucleotide.

Non-limiting examples of nucleic acid molecules that may be included in the compositions described above also include, DNA, such as genomic or cDNA; RNA, such as mRNA, sRNA or rRNA; or a hybrid of DNA and RNA. The composition may further comprise a labelled-nucleotide.

The term “salt” may include salts prepared from toxic or non-toxic acids or bases including inorganic acids and bases and organic acids and bases. Salts may be prepared from, for example, pharmaceutically acceptable non-toxic acids including inorganic and organic acids.

Any surfactant known to one skilled in the art may be also be included in the composition, particularly, when the composition is lyophilised. The surfactant may be polyionic, non-ionic, or ionic (specifically cationic or anionic), or may be zwitterionic. A surfactant as described herein includes Tween-20, Tween 80, CHAPS, or other detergent such as Brij-L23, Pluronic-F127, or a combination thereof. Examples of suitable surfactants include but are not limited to polyacrylate surfactants, silicone surfactants, and/or other commercially available surfactants or detergents. The composition described herein may include an anionic surfactant which contains an anionic functional group at one end, such as a sulfate, sulfonate, phosphate, and carboxylate functional group. The reagent may comprise a neutral surfactant, for example, a polyethelene glycol lauryl ether.

Sample preparation reagents as described herein may include, for example, lysis buffer, proteinase K (PK1), purification beads (PB), resuspension buffer (RSB), and ethanol (EtOH). Library preparation reagents as described herein may include, for example, end repair mix, A-Tailing mix, ligation mix, unique molecular identifiers (UMI), stop ligation buffer, as well as Tag buffers, nicotinamide-adenine dinucleotide (NAD⁺), ligase, indexes, beads, SDS, switching oligos, dNTPs, and buffers.

The composition may further, or in the alternative, include an enzyme inhibitor, a molecular probe, a crowding agent, organic osmolite, cyclodextrin, adenosine triphosphate (ATP), ethylenediaminetetraacetic acid (EDTA), creatine kinase, creatine phosphate, palladium, lipoic acid, hexaethylene glycol, trihydroxypropanephosphine, sodium ascorbate, or any combination thereof. An enzyme inhibitor as described herein includes any a molecule that binds to an enzyme and decreases its activity. A molecular probe as described herein includes, for example, digoxigenin, 8-Anilinonaphthalene-1-sulfonic acid (“ANS”), porphyrin, BODIPY, cyanine, or any combination thereof. A crowding agent as described herein includes any crowding agent known to those skilled in the art. Examples include, but are not limited to, polyethylene glycol, ficoll, dextran, and serum albumin. In one implementation, the composition includes about 5 wt. %, about 4 wt. 5%, about 3 wt. 5, about 2 wt. %, about 1 wt. %, less than about 1 wt. % of a crowding agent, for example, less than about 0.001 wt. %, about 0.001 wt. %, about 0.005 wt. %, about 0.01 wt. %, about 0.05 wt. %, about 0.1 wt. %, about 0.5 wt. %, about 1 wt. % of an additional compound, or any amount or range therebetween. In one implementation, there is no measurable content of crowding agent in the composition.

Those skilled in the art of sequencing technologies will appreciate there are additional reagents that may be useful in the compositions, methods, kits, cartridges, and systems of the present disclosure that are not explicitly described herein.

The composition described herein may further include a water purification compound. A water purification compound as described herein includes any compound that may be used to purify water, for example, a compound that removes or renders inert undesirable chemicals from water to prepare that water for sequencing applications. A water purification compound as described herein allows for the use of atmospheric water capture technology to reduce cartridge size for sequencing applications, as well as a reduction in environmental impact by reducing or eliminating water shipped with or in the cartridge since water is collected on the instrument. A water purification compound as described herein resolves water quality issues that are otherwise associated with atmospheric water capture. Likewise, the water purification compound as described herein may allow for the use of other non-purified water sources such as, for example, municipal water sources, ground water, and reclaimed or recycled water sources.

Water purification compounds including, for example, tablets, may, in one implementation, be incorporated into a sequential workflow, which may take multiple forms. For example, in one implementation, a large, single water purification tablet may be used in in the tank where the atmospheric-captured water is stored. Alternatively, a small water purification tablet may be incorporated into each composition that includes one or more reagent and of a size proportionate to the amount of liquid that capsule will utilize for rehydration. In one implementation, the water purification compound comprises sodium dichloroisocyanurate, chlorine, chloramines, chlorine dioxide, polyaluminium chloride, aluminum sulfate, ferric sulfate, hydrogen peroxide, sodium hydroxide bromide, silver nanoparticles, iron, iodine, activated carbon, or any combination thereof. In one implementation, the water purification compound is in a position between the shell and the interior compartment. Alternatively, the water purification compound may be on or a part of the shell in other implementations.

Sodium dichloroisocyanurate (NaDCC) and other water purification compounds may not be compatible with reagents such as those used for sequencing applications, or as used in sequencing applications generally. NaDCC's mechanism of action generates hypochlorous acid which is lethal to microorganisms by inhibiting DNA replication, causing oxidation, causing protein aggregation and in general causing inactivation of enzymes/proteins. The compositions, methods, cartridges, and systems described herein address this problem by using the delayed release aspect of the design described herein. In one implementation, water is added to a cartridge containing reagents fitted with a water purification compound, the water purification compound dissolves, water purification takes place via hypochlorous acid release damaging microorganisms, hypochlorous acid is stopped, a composition containing one or more reagent releases, the one or more reagent dissolves, and the reagent mix is ready to use.

The compositions, methods, cartridges, and systems described herein may include, in the interior compartment, one or more dry reagent, one or more microsphere, one or more bead, one or more powder, one or more cake, one or more gel, one or more liquid, or any combination thereof.

The compositions and reagents described herein may include dry reagents and may optionally be lyophilised as, for example, a lyophilised microsphere. In one implementation, the composition includes a cake, a bead, or a powder. In another implementation, the composition may be a microsphere, a cake, or a combination thereof.

When the composition is in the form of a cake or a bead (e.g., a microsphere), the composition may exhibit mechanical rigidity. “Mechanical rigidity” of a bulk composition (for example, of a cake or bead) as used herein refers to a bulk composition that exhibits a loss of mass of up to 5%, more preferably up to 1%, even more preferably up to 0.5%, and most preferably up to 0.1% from the bulk composition after the bulk composition is subjected to mechanical stress such as vibration or shock stress. Maintaining mechanical rigidity of a bulk composition helps to reduce or prevent the loss of a lyophilised material during shipping. If, for example, a cake or a bead lacks mechanical stability, incomplete rehydration may occur, resulting in a loss of efficiency in a sequencing reaction. Incomplete rehydration could be caused by the unpredictable position of the lyophilized material where lyo fragments or shed powders might be located beyond the line of rehydration.

As used herein, “microsphere” includes spherical particles or beads that have a diameter of 0.1 μm to 25,000 μm. For example, a microsphere may have a diameter of about 0.1 μm, 0.5 μm, 1 μm, 10 μm, 20 μm, 25 μm, 30 μm, 35 μm, 40 μm, 50 μm, 60 μm, 70 μm, 80 μm, 90 μm, 100 μm, 150 μm, 200 μm, 300 μm, 400 μm, 500 μm, 600 μm, 700 μm, 800 μm, 900 μm, 1000 μm, 10,000 μm, 25,000 μm, or any diameter between about 0.1 μm and about 25,000 μm. In one implementation, the microsphere has a diameter between about 100 μm and about 1000 μm. In one implementation, the microsphere has a cross-section of between about 0.1 mm and about 25 mm. In one implementation, the microsphere has a cross-section of between about 0.1 mm and about 1 mm. In one implementation, the composition has a cross-section of greater than about 1 mm. In one implementation, the composition has a diameter of about 0.1 mm, 1 mm, 2 mm, 3 mm, 4 mm, 5 mm, 6 mm, 7 mm, 8 mm, 9 mm, 10 mm, 50 mm, 100 mm, 200 mm, 300 mm, 400 mm 500 mm, 600 mm, 700 mm, 800 mm, 900 mm, 1,000 mm, or any diameter between about 0.1 mm and about 1,000 mm.

In one implementation, the microsphere is spherical, elliptical, or toroidal. Microspheres are generally comprised of an outer polymer layer and may include one or more of the shell ingredients described herein. Microspheres may include, for example, biodegradable polymers. Microspheres in accordance with the present disclosure include those prepared by conventional techniques, which are known to those skilled in the art. For example, microspheres may be prepared by freezing a liquid into frozen pellets, followed by placing frozen microspheres in a dryer, for example, dried by heat or in tray lyophilisers such as a conventional tray dryer, or in a rotational dryer. In the present disclosure, the term “lyophilize” or “lyophilizate” will be used as equivalent terms of “lyophilised”, “lyophilizate”, or “freeze-dried” e.g., with respect to a compositions, methods, cartridges, and systems described herein. Microencapsulation as described herein includes the coating of individual microspheres or particles in one or more powder.

Macrospheres in accordance with the present disclosure include those prepared by conventional techniques, which are known to those skilled in the art. The compositions, methods, cartridges, and systems described herein may include a single microsphere, or may include a plurality of microspheres and may thereby form a macrosphere. For example, the composition described herein may include anywhere between 1 and over 1,000,000 microspheres. In one implementation, the composition includes 1 microsphere, or less than 25 microspheres, or less than 50 microspheres, or less than 75 microspheres, or less than 100 microspheres, or less than 500 microspheres, or any number of microspheres between about 1 and about 1,000,000. In one implementation, for example in a macrosphere, compositions and/or reagents are different. Macroencapsulation as described herein includes the coating of a plurality of microspheres or particles in one or more powder. In one implementation, one or more macrosphere as described herein may be coated for timed release.

In one implementation, the interior compartment includes a plurality of microspheres comprising a plurality of reagents. In another implementation, the interior compartment includes a plurality of microspheres comprising one reagent. As described herein, each of the plurality of microspheres may include a plurality of reagents. Alternatively, the plurality of microspheres may collectively include a plurality of reagents.

Lyophilisable formulations can be reconstituted into solutions, suspensions, emulsions, or any other suitable form for administration or use. Lyophilisable formulations are typically first prepared as liquids, then frozen and lyophilised. The total liquid volume before lyophilisation can be less than, equal to, or more than, the final reconstituted volume of the lyophilised formulation. The final reconstituted volume of the lyophilised formulation may be less than the total liquid volume before lyophilisation, or may be greater than the total liquid volume before lyophilisation, or may be an equivalent total liquid volume to before lyophilisation.

Lyophilised formulations can be stored at a wide range of temperatures. Lyophilised formulations may be stored below 25° C., for example, refrigerated at 2-8° C., or at room temperature (e.g., approximately 25° C.). Lyophilised formulations may be stored at about 0° C., 5° C., 10° C., 15° C., 20° C., 25° C., 30° C., 35° C., 37° C., or any temperature between 37° C. and −80° C. For example, they compositions may be stored between about 15° C. and about 37° C., below about 25° C., at about 4-20° C.; below about 4° C.; below about −20° C.; about −40° C.; about −70° C., or about −80° C. Stability of the lyophilised formulation may be determined in a number of ways known in the art, for example, by visual appearance of the composition and/or cake and/or by moisture content. The compositions of the present disclosure can also withstand temperature excursions that might occur during shipping, for example, up to 70° C. The compositions, methods, cartridges, and systems described herein, in one implementation, exhibit stability when stored for a period of time, for example, 10 days, 14 days, 20 days, 26 days, 30 days, 60 days, 100 days, 200 days, 300 days, 365 days, or more when stored at a temperature of 37° C. for example.

Lyophilised formulations are typically rehydrated (interchangeably referred to herein as “reconstituted”) for use by addition of an aqueous solution to dissolve the lyophilised formulation. A wide variety of aqueous solutions can be used to reconstitute a lyophilised formulation including water, saline, or another electrolyte or non-electrolyte diluent. It may be preferable in certain circumstances that the lyophilised compositions described herein are reconstituted using water. Lyophilised formulations may be rehydrated with a solution comprising water (e.g., USP WFI, or water for injection) or bacteriostatic water (e.g., USP WFI with 0.9% benzyl alcohol). However, solutions comprising additives, buffers, excipients, and/or carriers can also be used.

Freeze-dried or lyophilised formulations are typically prepared from liquids, that is, from solutions, suspensions, emulsions, and the like. Thus, the liquid that is to undergo freeze-drying or lyophilisation may include all components desired in a final reconstituted liquid formulation. Alternatively, the liquid that is to be lyophilised may include a single reagent, then, once lyophilised, be dry compounded together with one or more additional lyophilised reagents such that those reagents are mixed together upon rehydration to form the reconstituted liquid formulation. Accordingly, one lyophilised material may be rehydrated, or, two or more lyophilised materials may be rehydrated together. As a result, when rehydrated or reconstituted, the freeze-dried or lyophilised formulation will render a desired liquid formulation upon reconstitution.

In one implementation, the compositions described herein, when lyophilised, include a moisture content of below about 10 wt. %. For example, the moisture content may be less than about 9.5 wt. %, less than about 9 wt. %, less than about 8.5 wt. %, less than about 8 wt. %, less than about 7.5 wt. %, less than about 7 wt. %, less than about 6.5 wt. %, less than about 6 wt. %, less than about 5.5 wt. %, less than about 5 wt. % water, less than about 4.5 wt. %, less than about 4 wt. %, less than about 3.5 wt. %, less than about 3 wt. %, less than about 2.5 wt. %, less than about 2 wt. %, less than about 1.5 wt. %, less than about 1 wt. %, less than about 0.5 wt. %, less than about 0.1 wt. % water, or any amount therebetween. In one implementation, there is no measurable content of water in the lyophilised composition.

The composition may be any appropriate size or volume that is appropriate to encapsulate one or more reagents and suitable for use in library preparation for sequencing. In one implementation, the composition has a volume of reagent in the core region of between about 0.1 μL, and about 500 μL. For example, the composition may have an active reagent volume of about 0.1 μL, 0.5 μL, 1 μL, 2 μL, 3 μL, 4 μL, 5 μL, 6 μL, 7 μL, 8 μL, 9 μL, 10 μL, 15 μL, 20 μL, 25 μL, 30 μL, 35 μL, 40 μL, 45 μL, 50 μL, 60 μL, 70 μL, 80 μL, 90 μL, 100 μL, 125 μL, 150 μL, 175 μL, 200 μL, 225 μL, 250 μL, 275 μL, 300 μL, 325 μL, 350 μL, 375 μL, 400 μL, 425 μL, 450 μL, 475 μL, 500 μL, or any volume between about 0.1 μL, and about 500 μL. For example, the active reagent volume may be between about 10 μL, and about 400 μL, between about 100 μL, and about 500 μL, between about 200 μL, and about 500 μL, between about 300 μL, and about 500 μL, between about 400 μL, and about 500 μL, between about 0.1 μL, and about 100 μL, or between about 0.1 μL, and about 500 μL.

The compositions described herein may include an additional reagent in the shell. In one implementation, the composition includes a reagent or additive in the shell. The reagent in the shell may include, for example, any of the foregoing reagents or additives. In one implementation, the shell contains no nucleic acid molecules, for example, the shell contains no DNA. In one implementation, the shell contains more than one reagent and, or in the alternative, more than one additive.

The compositions described herein may be used for multiple sequential co-assays comprising lysis, DNA analysis, RNA analysis, protein analysis, tagmentation, nucleic acid amplification, nucleic acid sequencing, DNA library preparation, SBS technology, assay for transposase accessible chromatic using sequencing (ATAC-seq), contiguity-preserving transposition (CPT-seq), single cell combinatorial indexed sequencing (SCI-seq), or single cell genome amplification, or any combination thereof performed sequentially. In one implementation, the composition is used for performing multiple co-assay reactions. The compositions, methods, cartridges, and systems described herein may, in one implementation, improve sequencing quality, enable one-pot library prep, and simplify manufacturing and use. As used herein, the term “one-pot reaction” may also be referred to as “transfer-free reaction.”

The compositions, methods, cartridges, and systems described herein may be prepared for various stages of sequencing including, but not limited to, sample extraction, library preparation, enrichment, clustering, and sequencing. The composition may include any number of different reagents from those described herein or any reagent that may be useful in promoting utility of sequencing systems, for example, SBS technology.

In one implementation, a biological sample contacts the composition. A biological sample, may include, for example, whole blood, lymphatic fluid, serum, plasma, sweat, tear, saliva, sputum, cerebrospinal fluid, amniotic fluid, seminal fluid, vaginal excretion, serous fluid, synovial fluid, pericardial fluid, peritoneal fluid, pleural fluid, transudates, exudates, cystic fluid, bile, urine, gastric fluid, intestinal fluid, fecal samples, liquids containing single or multiple cells, liquids containing organelles, fluidized tissues, fluidized organisms, liquids containing multi-celled organisms, biological swabs and biological washes. A biological sample can include nucleic acids, such as DNA, genomic DNA, RNA, mRNA or analogs thereof; nucleotides such as deoxyribonucleotides, ribonucleotides or analogs thereof such as analogs having terminator moieties such as those described in Bentley et al., “Accurate Whole Human Genome Sequencing Using Reversible Terminator Chemistry,” Nature 456:53-59 (2008) and WO/2013/131962, which are hereby incorporated by reference in their entirety.

A second aspect relates to a composition. The composition includes a dissolvable first shell, and a dissolvable second shell, the second shell comprising one or more reagent.

This aspect may be in accordance with the previously described aspect.

In one implementation, the first shell dissolves when the composition is exposed to a first release condition. In one implementation, the second shell prevents release of the one or more reagent when the composition is exposed to the first release condition. In another implementation, the second shell dissolves when exposed to a second release condition. In one implementation, the first shell is an outer shell. In one implementation, the second shell is an interior shell.

In one implementation, the first shell dissolves when the first shell is exposed to at least one additional first shell release condition, where one or more of the at least one additional first shell release condition is different from the first release condition. In another implementation, the second shell prevents release of the one or more reagent when the second shell is exposed to the at least one additional first shell release condition. In one implementation, the second shell releases the one or more reagent when the second shell is exposed to at least one additional second shell release condition, where one or more of the at least one additional second shell release condition is different from the second release condition. The additional first shell release condition and the additional second shell release condition may be in addition, or in the alternative, to the first and second release condition.

In one implementation, the first shell has a first shell width and the second shell has a second shell width, and the first shell width is different from the second shell width. In another implementation, the first shell width is between about 1 micrometer and about 1,000 micrometers. In yet another implementation, the second shell width is between about 1 micrometer and about 1,000 micrometers.

In one implementation, the first shell comprises a water-soluble compound. In another implementation, the first shell comprises one or more of polyvinyl alcohol, polyvinylpyrrolidone (PVP), carrageenan, gelatin, hydroxypropyl methylcellulose (HPMC), pullulan, starch film, benzoxaborole-poly(vinyl alcohol) (benzoxaborole-PVA), pectin, or any combination thereof, as described in the previous aspect.

In one implementation, the second shell comprises one or more dry reagent, one or more microsphere, one or more bead, one or more powder, one or more cake, one or more gel, one or more liquid, or any combination thereof. In another implementation, the one or more reagent is lyophilised. In one implementation, the second shell comprises a plurality of microspheres comprising a plurality of reagents. In another implementation, the second shell comprises a plurality of microspheres comprising one reagent.

A third aspect relates to a composition. The composition includes a dissolvable first shell; a dissolvable second shell, the second shell comprising one or more reagent; and a water purification compound.

This aspect may be in accordance with the previously described aspects.

In one implementation, the water purification compound is in a position between the dissolvable first shell and the dissolvable second shell. In one implementation, the first shell is an outer shell. In one implementation, the second shell is an interior shell.

A fourth aspect relates to a method for controlling release of one or more reagent. The method includes providing a composition comprising a shell surrounding an interior compartment, wherein said interior compartment comprises one or more reagent; exposing said composition to a first release condition to release said interior compartment; and exposing said interior compartment to a second release condition to release said one or more reagent, wherein said first release condition is different from said second release condition.

This aspect may be carried out in accordance with the previously described aspects.

In one implementation, the first release condition comprises a pH of between about 1.0 and about 10.0. In another implementation, the second release condition comprises a pH of between about 1.0 and about 10.0. For example, the first release condition or second release condition may include a pH that is less than 3. Alternatively, the first release condition or second condition may include a pH that is above 5 or 7 or 8, depending on materials used. In one implementation, the second release condition is effective to release a plurality of reagents, where the content of at least one reagent is different from the content of at least one other reagent. In one implementation, exposing the shell to the first release condition and exposing the interior compartment to the second release condition occurs sequentially.

In one implementation, a pH in the rehydration solution is between about 1.0 and about 10.0. A pH of the rehydration solution may be, for example, about 1.0, about 2.0, about 3.0, about 4.0, about 5.0, about 6.0, about 7.0, about 7.5, about 8.0, about 8.5, about 9.0, about 9.5, about 10.0, or any amount therebetween. Rehydration time will vary depending on composition content and reaction conditions (e.g., reagents, temperature, pH) as described herein. In one implementation, rehydration time may be between 0.1 seconds and 10 hours. For example, rehydration time may be about 0.1 seconds, 1 second, 10 seconds, 30 seconds, 45 seconds, 60 seconds, 5 minutes, 10 minutes, 12 minutes, 15 minutes, 20 minutes, 30 minutes, 40 minutes, 50 minutes, 60 minutes, 70 minutes, 80 minutes, 90 minutes, 2 hours, 5 hours, 8 hours, 10 hours, or any amount of time therebetween.

A rehydration (or reconstitution) solution as used herein may include water, deionized water, saline solutions, acidic solutions, basic solutions, detergent solutions and/or buffers, and may be in accordance with rehydration solutions previously described. In one implementation, the rehydration solution is water, ethanolamine, or a combination thereof. In one implementation, reagents described herein having varying concentrations, types of enzymes, and different amounts of co-factors, salts, pHs, and more, can be rehydrated with water alone, or even atmospheric water capture. Additional additives as described herein may be provided in the rehydration solution to further improve control of release of microspheres.

In one implementation, the method further includes using the rehydrated composition in a sequencing by synthesis process. In another implementation, the method further includes exposing the rehydrated composition to a sequencing primer, where incorporation of the one or more modified nucleotide in the sequencing primer generates an extended sequencing primer. In another implementation, the method further includes applying the rehydrated composition to a solid support comprising a nucleotide cluster, where the nucleotide cluster comprises a target polynucleotide.

A fifth aspect relates to a method for controlling release of one or more reagent. The method includes providing a composition comprising: a dissolvable first shell, and a dissolvable second shell, the second shell comprising one or more reagent; exposing said composition to a first release condition to dissolve said first shell; and exposing said composition to a second release condition to dissolve said second shell, wherein said first release condition is different from said second release condition.

This aspect may be carried out in accordance with the previously described aspects.

A sixth aspect relates to a method for controlling release of one or more reagent. The method includes providing a composition comprising: a dissolvable first shell, a dissolvable second shell, the second shell comprising one or more reagent, and a water purification compound; exposing said composition to a first release condition to dissolve said water purification compound; exposing said composition to a second condition to dissolve said first shell; and exposing said composition to a third release condition to dissolve said second shell, wherein said first release condition is different from said second release condition.

This aspect may be carried out in accordance with the previously described aspects.

In one implementation, the first shell is an exterior shell. In another implementation, the second shell is an interior shell.

A seventh aspect relates to a method. The method includes providing a capsule in a well at a first temperature; providing a liquid having a temperature in said well; elevating the temperature of the liquid to a second temperature; lowering the temperature of the liquid from the second temperature to a third temperature; and releasing one or more reagents from said capsule.

This aspect may be carried out in accordance with the previously described aspects.

In one implementation, the capsule comprises a composition comprising a shell surrounding an interior compartment, where the interior compartment comprises one or more reagent and where the shell releases the interior compartment when the shell is exposed to a first release condition, where the interior compartment releases the one or more reagent when the interior compartment is exposed to a second release condition, and where the first release condition is different from the second release condition.

In one implementation, the first temperature is different from the third temperature. In another implementation, the first temperature is the same as the third temperature.

An eighth aspect relates to a method. The method includes dissolving an exterior shell of a capsule in a well at a first temperature, where the well comprises a liquid, where the capsule comprises the exterior shell, a water purification compound, an interior shell, and one or more reagent, where dissolving the exterior shell of the capsule releases the water purification compound; elevating the temperature of the well to a second temperature; and dissolving the interior shell thereby releasing one or more reagent.

This aspect may be carried out in accordance with the previously described aspects.

In one implementation, dissolving the exterior shell of the capsule in the well comprises flowing the liquid into the well. In another implementation, dissolving the interior shell comprises raising the pH of the liquid above 7.0. In another implementation, dissolving the interior shell comprises lowering the pH of the liquid below 7.0. In yet another implementation, the interior shell is dissolved by the second temperature. In another implementation, the interior shell is dissolved after a minimum time period. In one implementation, the minimum time period is 5 minutes.

A ninth aspect relates to a cartridge. The cartridge includes a reagent reservoir, wherein the reagent reservoir comprises a composition, said composition comprising: a shell surrounding an interior compartment, wherein said interior compartment comprises one or more reagent and wherein said shell releases said interior compartment when said shell is exposed to a first release condition, wherein said interior compartment releases said one or more reagent when said interior compartment is exposed to a second release condition, and wherein said first release condition is different from said second release condition.

This aspect may be in accordance with the previously described aspects.

In one implementation, the first release condition is exposure to a liquid.

In one implementation, the second release condition is exposure to a temperature above about 25° C.

Exemplary cartridges and configurations are described in, for example, U.S. Pat. No. 8,637,242, which is hereby incorporated by reference in its entirety. Exemplary flow cells are described, for example, in U.S. Pat. No. 8,241,573, which is hereby incorporated by reference in its entirety.

Additionally or alternatively, a cartridge can include separate reservoirs and fluidic systems used to carry out amplification methods and to carry out detection methods. Examples of integrated sequencing systems that are capable of creating amplified nucleic acids and also determining the sequence of the nucleic acids include, without limitation, the MiSeg™ platform (Illumina, Inc., San Diego, Calif.) and devices described in U.S. Pat. No. 8,951,781, which is hereby incorporated by reference in its entirety.

A tenth aspect relates to a cartridge. The cartridge comprises a reagent reservoir, wherein the reagent reservoir comprises a composition, said composition comprising: a dissolvable first shell, and a dissolvable second shell, the second shell comprising one or more reagent.

This aspect may be in accordance with the previously described aspects. In one implementation, the first shell is an exterior shell. In one implementation, the second shell is an interior shell.

An eleventh aspect relates to a system for controlling release of one or more reagent. The system includes a well; a composition comprising: a shell surrounding an interior compartment, wherein said interior compartment comprises one or more reagent, and wherein said shell releases said interior compartment when said shell is exposed to a first release condition, wherein said interior compartment releases said one or more reagent when said interior compartment is exposed to a second release condition, and wherein said first release condition is different from said second release condition; and a liquid.

This aspect may be in accordance with the previously described aspects.

The liquid as described herein may be present in the well or, alternatively, the composition may be present in the well. In one implementation, the liquid is in the well. In another implementation, the composition is in the well.

The system may further include a temperature controller or sensor. The temperature controller may be used to change or adjust temperature of the system to further control release of various components of the compositions described herein. For example, the temperature controller may be used to speed up or slow down the release of the shell or dissolvable exterior shell. Similarly, the temperature controller may be used to speed up or slow down the release of the interior compartment or dissolvable interior shell to facilitate or control the release of one or more reagents. In one implementation, the system comprises a temperature controller on the well. For example, the temperature controller may include a resistive heater proximate to a wall of the well to provide heat thereto. The temperature controller may also include a temperature sensor. The temperature controller may also include circuitry to activate and deactivate the heater to maintain the well at a specified temperature.

A twelfth aspect relates to a system for controlling release of one or more reagent. The system includes a well; a composition comprising: a dissolvable first shell, and a dissolvable second shell, the second shell comprising one or more reagent; and a liquid.

This aspect may be in accordance with the previously described aspects.

In one implementation, the first shell is an exterior shell. In one implementation, the second shell is an interior shell.

A thirteenth aspect relates to a method. The method includes: flowing a liquid having a temperature into a well, where the well comprises a capsule, where the capsule comprises a first shell surrounding a water purification compound and a second shell surrounding one or more reagent, wherein said first shell releases said water purification compound upon exposure to a first release condition, wherein said second shell releases said one or more reagent upon exposure to a second release condition, wherein said first release condition is different from said second release condition, wherein said water purification compound substantially or completely degrades upon exposure to a degradation condition; exposing said first shell to the first release condition whereby the water purification compound is released; exposing said water purification compound to the degradation condition whereby said water purification compound is substantially or completely degraded; and exposing said second shell condition to the second release condition whereby said one or more reagent is released.

This aspect may be carried out in accordance with the previously described aspects.

In one implementation, the first release condition is exposure to the liquid. In another implementation, the degradation condition is an elevated temperature of the liquid. In one implementation, the elevated temperature is greater than or equal to about 25° C. In one implementation, the degradation condition is the same as the second release condition. In one implementation, flowing a liquid, exposing said first shell to the first release condition, and exposing said water purification compound to the degradation condition are performed in order. In another implementation, flowing a liquid, exposing said first shell to the first release condition, exposing said water purification compound to the degradation condition, and exposing said second shell condition to the second release condition are performed in order.

It should be appreciated that all combinations of the foregoing concepts and additional concepts discussed in greater detail herein (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

In the present disclosure, reference is made to the accompanying drawings that form a part hereof, and in which is shown by way of illustration specific implementations which may be practiced. These implementations are described in detail to enable those skilled in the art to practice the disclosure, and it is to be understood that other implementations may be utilized and that structural, logical and electrical changes may be made without departing from the scope of the present disclosure. The following description of example implementations is, therefore, not to be taken in a limited sense.

The present disclosure may be further illustrated by reference to the following examples.

### EXAMPLES

The following examples are intended to illustrate, but by no means are intended to limit, the scope of the present disclosure as set forth in the appended claims.

### Example 1—Water Purification Compound for Reagents

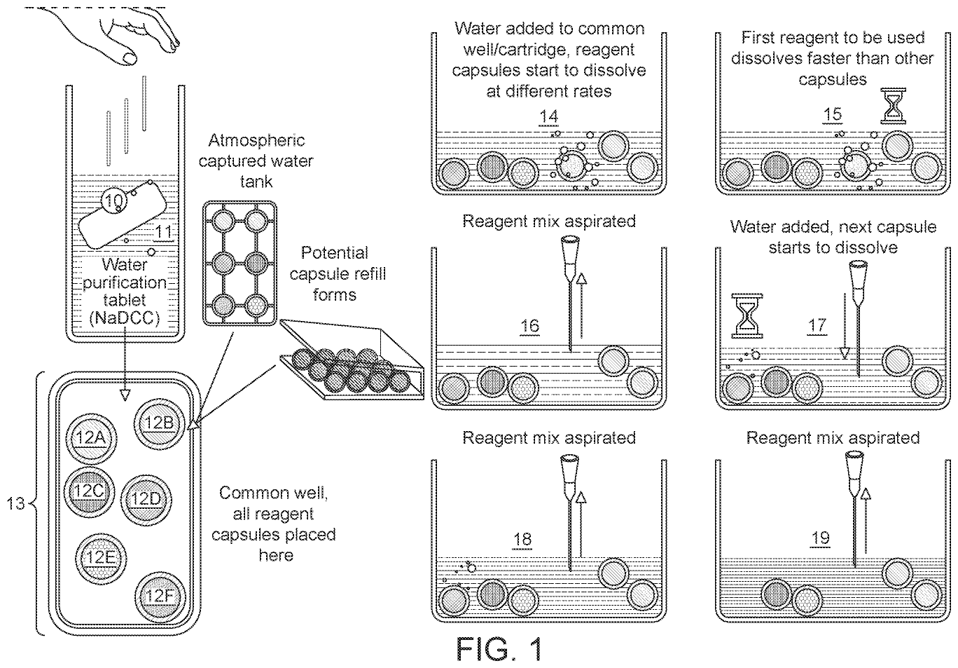

The use of water-soluble film casings to encapsulate reagents adds value and can shorten and simplify workflows. This may be exploited to further simplify both workflows and the operation of a sequencer at large through encapsulating different reagents, for example sequencing reagents, in different films with different dissolving times and release triggers. The sequencer/cartridge architecture may move towards just one common well with capsules of all the sequencing reagents inside, rather than individual wells for each reagent. Many approaches can be taken to differentiate the dissolving profile of the various reagents, including, film composition, film thickness, release triggers (pH, light, temperature, time), and capsule design. This sequential release capsule technology can be combined with atmospheric water capture technology to further reduce cartridge size, as well as environmental impact in terms of eliminating the water (and associated packaging) for worldwide shipping. However, this would mandate the potential water quality issues associated with atmospheric water capture being addressed. In addition to being managed at the local level (i.e. by columns, filters), water purification tablets can be incorporated into the sequential workflow. This could take multiple forms, either a grand water purification tablet in the tank the atmospheric-captured water is stored, or a small tablet incorporated into each reagent capsule and of a size proportionate to the amount of liquid that capsule will rehydrate to as shown in FIG. 1.

FIG. 1 shows a water purification compound in sequential workflow according to selected implementations of the current disclosure. A water purification compound 10 is placed into an atmospheric water tank 11. Reagent capsules 12A-12F are placed in a well 13. Water from the atmospheric water tank is added to the well, as shown in 14. A first reagent capsule dissolves faster than the other reagent capsules, as shown in 15, thereby dissolving the first reagent first. In some implementations, mixing may occur. The dissolved first reagent is then aspirated, as shown in 16. More water is added to the well, as shown in 17, and a second reagent capsule dissolves. The second capsule may dissolve in water slower than the first reagent capsule, or alternatively, may dissolve upon another release condition, such as upon exposure to light. Once the second capsule is fully dissolved, it may be aspirated, as shown in 18. Subsequent reagent capsules may be dissolved and aspirated, as shown in 19, for example, upon exposing the other reagent capsules to other release conditions.

Light may be used as at least one of the release conditions. For example, exposing a capsule to light may cause the capsule (or shell of the capsule) to dissolve. In some implementations, the release condition may be any or all wavelengths of light. In other implementations, the release condition may be a specific wavelength or range of wavelengths. For example, a first release condition may include exposure of a first range of wavelengths, and a second release condition may include exposure of a second range of wavelengths, where the first range of wavelengths and the second range of wavelengths do not overlap.

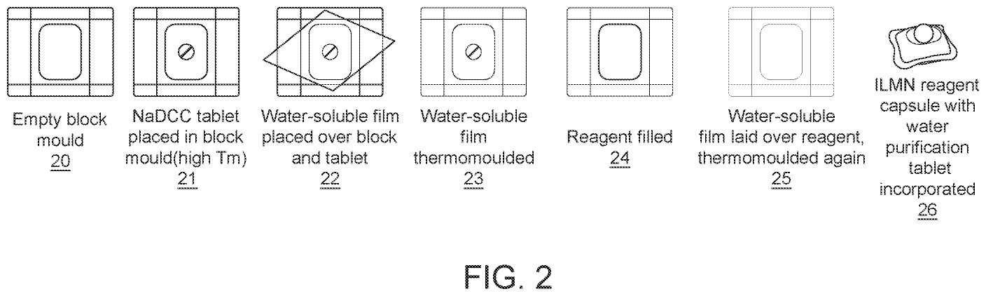

A chemical that may be used for water purification in the compositions, methods, cartridges, and systems described herein is sodium dichloroisocyanurate. This has a melting temperature of 255° C., so therefore can be incorporated into the moulding/manufacturing of the capsules as shown in FIG. 2. An empty block mould may be provided 20. A sodium dichloroisocyanurate (NaDCC) tablet is placed in the block mold 21. A first water-soluble film is placed over the block and tablet 22, and then thermomoulded 23. A reagent is then filled into the mould over the first water-soluble film 24. Another water-soluble film, which may be the same or different material as the first water-soluble film, is placed over the reagent and thermomoulded 25. The resulting reagent capsule may then be removed from the block mould 26.

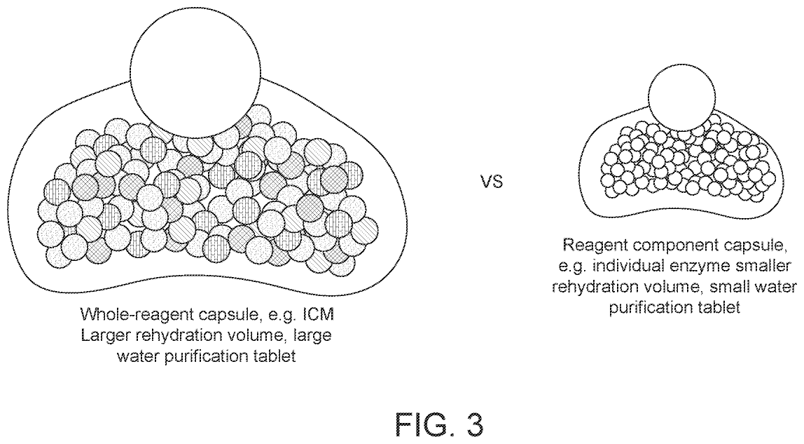

The size of the water tablet will scale with the rehydration volume/volume of the final reagent mix, as shown in FIG. 3.

### Example 2—Addressing Compatibility of Water Purification Compound with Reagents

An issue exists however regarding the compatibility of the sodium dichloroisocyanurate (NaDCC) tablet with reagents. NaDCC's mechanism of action is to generate hypochlorous acid. This acid is lethal to microorganisms by inhibiting DNA replication, causing oxidation, causing protein aggregation and in general causing inactivation of enzymes/proteins. This poses a clear incompatibility for reagents and workflows given the reliance on enzymes. Finetuning the water purification compound (e.g., NaDCC) can address this to reach a point where the microorganisms are killed but enzymes are unaffected. However, a more attractive approach is to use the delayed release aspect of the capsule design. In at least one such approach, water is added to cartridge containing reagents fitted with water purification tablets (e.g., NaDCC), the water purification tablet dissolves, water purification takes place via hypochlorous acid release damaging microorganisms, hypochlorous acid is stopped (for example, substantially or completed degraded or deactivated), a capsule containing reagent opens, the reagent begins to dissolve, and the reagent mix is ready to use.

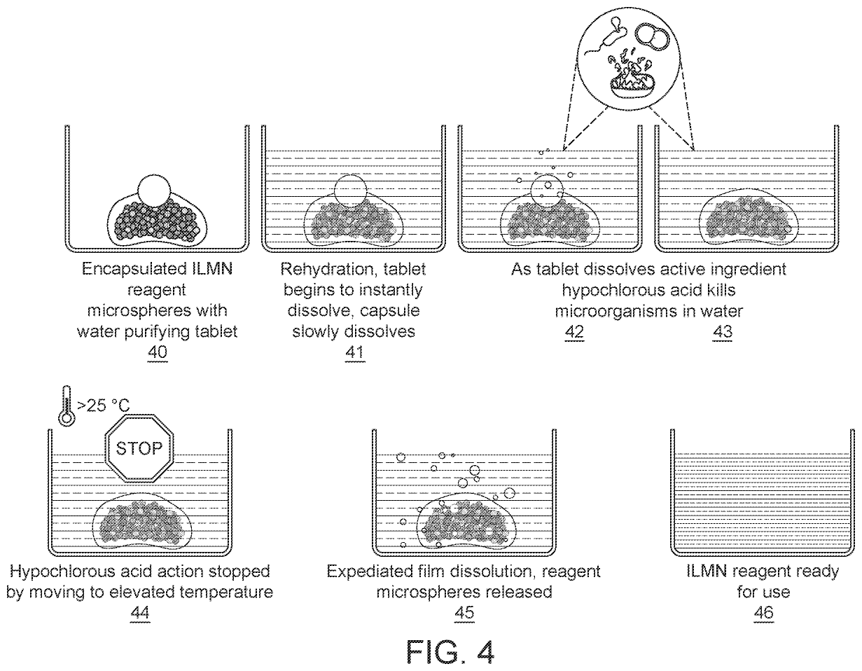

Hypochlorous acid is sensitive to or degraded by a number of things. For example, “HOCl is unstable against ultraviolet (UV) light, sunshine, contact with air, and elevated temperature (≥25° C.).” Ishihara et al., “Stability of Weakly Acidic Hypochlorous Acid Solution with Microbicidal Activity,” Biocontrol Science 22(4):223-227, abstract (2017), which is hereby incorporated by reference in its entirety. The presence of various organic compounds and inorganic ions results in rapid consumption of HOCl by oxidation reactions. The use of pure and cold water without contaminating compounds such as proteins and carbohydrates significantly reduce residual chlorine levels in both HOCl and ClO⁻ solutions. “[R]etention of HOCl levels appears to require formulation with pure water containing organic and inorganic compounds and ions at levels as low as possible.” These weaknesses can be exploited to develop the aforementioned system. A workflow of the present disclosure is exemplified in FIG. 4. A capsule containing reagent microspheres with a water purifying tablet is placed in a well 40. Upon rehydration, the tablet begins to instantly dissolve, while the capsule with the reagent microspheres dissolves at a slower rate 41. As the tablet dissolves, the active ingredient hypochlorous acid kills or disables microorganisms in the water 42, 43. Other water purification compounds may also be provided, for example, in the tablet, and help remove or disable other harmful chemicals or other substances. The hypochlorous acid is stopped by elevating the temperature of the water in the well to, for example, 25° C. or higher 44. After the hypochlorous acid is stopped, the capsule (film or shell) is dissolved, and the reagent microspheres are released 45. The dissolution of the capsule may occur after a period of time after being exposed to water, or alternatively, may dissolve upon exposure to another release condition such as, for example, light, light of a particular wavelength or range of wavelengths, pH above or below a certain threshold, or temperature above or below a certain threshold. After the reagent microspheres are dissolved and homogenized (for example, via mixing or diffusion), the reagent may be ready for use 46.

### Example 3—Composition and Capsule Design

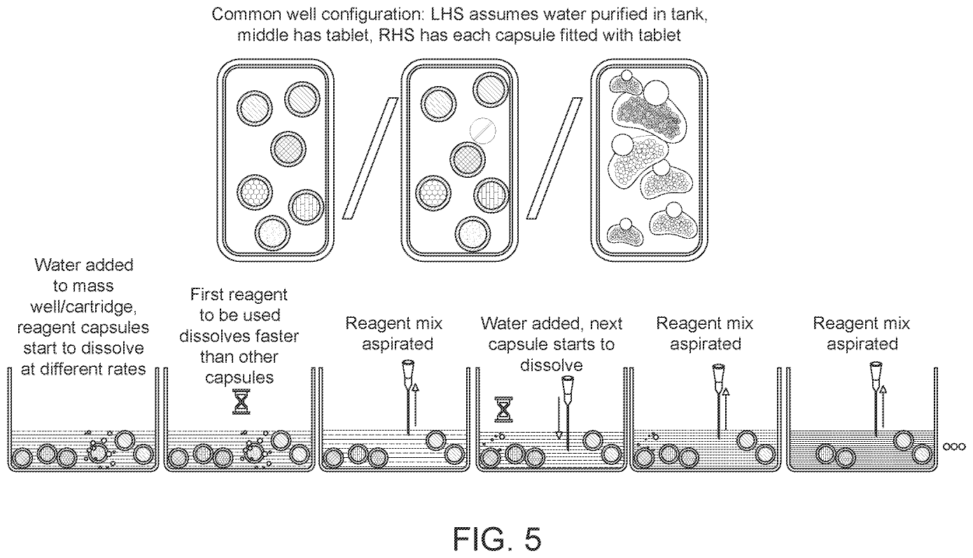

A capsule design can enable workflows such as those shown in FIG. 5. Packaging and utilizing sequencing reagents in this fashion would easily allow dosages to be altered. Not only could the number of capsules for each be simply multiplied (i.e., if for one run one capsule is used, two runs would use two capsules), large capsules could be created for high-throughput customers who currently pool many small (e.g., library) prep kits intended for individual runs into a pool large enough for a Hamilton robot to run 96 samples concurrently.

The above described setup will allow the same cartridge to be reused. A user may refill the cartridge by hand by dropping the refill capsules into the common well before starting a run. Alternatively, a robot or machine may dispense the appropriate refill capsules into the common well before starting a run. The capsule design also creates improvements in manufacturing and packaging where it makes dispensing easier due to the pre-dosed format.

An example of the compositions described herein are shown in FIG. 6, which has a shell 100 and an interior compartment 102. As noted herein, shell 100 may be referred to herein as a first shell or a dissolvable first shell or an exterior shell. Interior compartment 102 includes at least one reagent. As noted herein, interior compartment 102 may be referred to herein as a second shell or a dissolvable second shell and may include an interior shell. Shell 100 may, for example, release interior compartment 102 when the shell is exposed to a first release condition. Interior compartment 102 may, for example, release one or more reagent that is positioned inside interior compartment 102. As shown in FIG. 7, the composition may include a plurality of compositions, or may be used in conjunction with one or more additional compositions, which include a shell 100 (e.g., 100a, 100b, 100c, etc.) and an interior compartment 102 (e.g., 102a, 102b, 102c) with the different reagents, the same reagents, or substantially the same reagents. Furthermore, each of the outer shell 100 (e.g., 100a, 100b, 100c, etc.) and the interior compartment 102 (e.g., 102a, 102b, 102c) may respond to different release conditions, the same release conditions, or substantially similar release conditions.

An example of release of the compositions described herein is shown in FIG. 8 In FIG. 8, shell 100 surrounds interior compartment 102 and interior compartment 102 includes a plurality of reagents 104. The plurality of reagents may be different types of reagents and may be dry or substantially dry (e.g., lyophilised) as shown in FIG. 8. The composition, when placed in a first release condition, may dissolve shell 100 and release interior compartment 102. The composition, when placed in a second release condition, may dissolve interior compartment 102 and release one or more reagent 104. The first release condition may release a first reagent into a surrounding liquid environment. After a first reagent is released, the second release condition may release a second reagent as demonstrated in, for example, FIG. 8. In their current unencapsulated format, microspheres make on-board dosing unlikely due to static effects and the consequential impact on dosing accuracy. Using the water-soluble film means on-board metering is an option due to the microspheres being encapsulated and the static effects are minimized. Exemplary forms of microspheres that are useful in the present disclosure may be lyophilised and are shown in FIGS. 9 and 10.

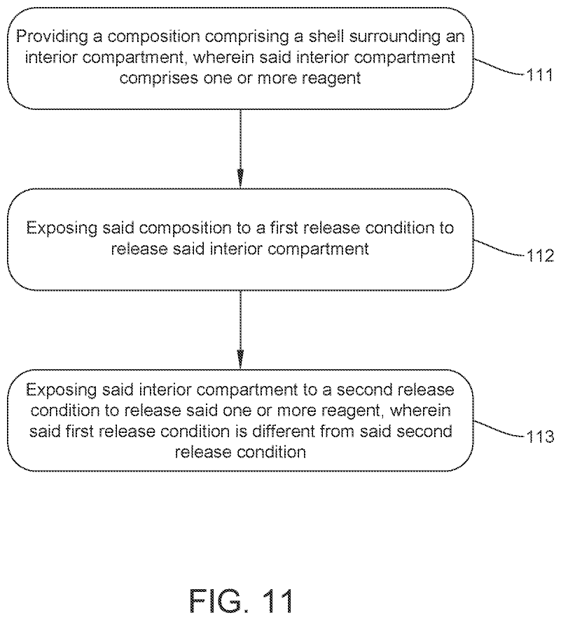

FIG. 11 is a flow chart describing one aspect described herein for a method for controlling release of one or more reagent. The method includes providing a composition comprising a shell surrounding an interior compartment, wherein said interior compartment comprises one or more reagent 111. The method further includes exposing said composition to a first release condition to release said interior compartment 112. The method further includes exposing said interior compartment to a second release condition to release said one or more reagent, wherein said first release condition is different from said second release condition 113.

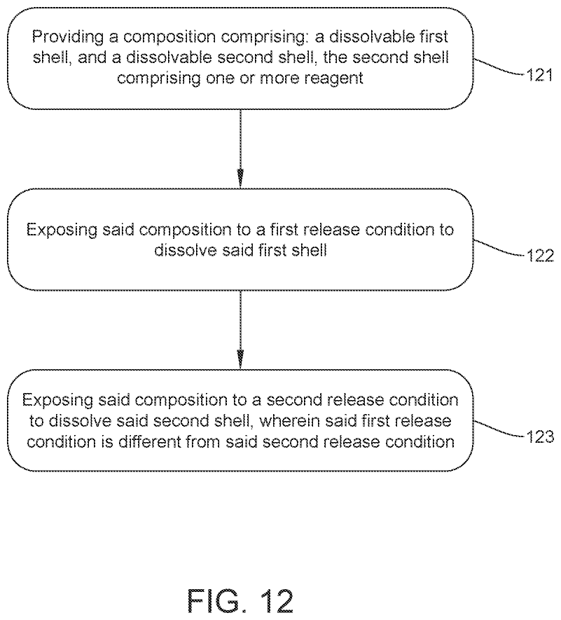

FIG. 12 is a flow chart describing one aspect described herein for a method for controlling release of one or more reagent. The method includes providing a composition comprising: a dissolvable first shell, and a dissolvable second shell, the second shell comprising one or more reagent 121. The method further includes exposing said composition to a first release condition to dissolve said first shell 122. The method further includes exposing said composition to a second release condition to dissolve said second shell, wherein said first release condition is different from said second release condition 123.

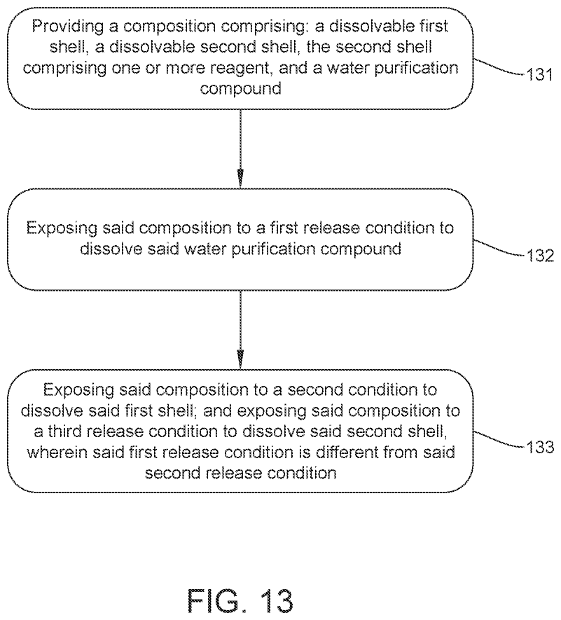

FIG. 13 is a flow chart describing one aspect described herein for a method for controlling release of one or more reagent. The method includes providing a composition comprising: a dissolvable first shell, a dissolvable second shell, the second shell comprising one or more reagent, and a water purification compound 131. The method further includes exposing said composition to a first release condition to dissolve said water purification compound 132. The method further includes exposing said composition to a second condition to dissolve said first shell; and exposing said composition to a third release condition to dissolve said second shell, wherein said first release condition is different from said second release condition 133.

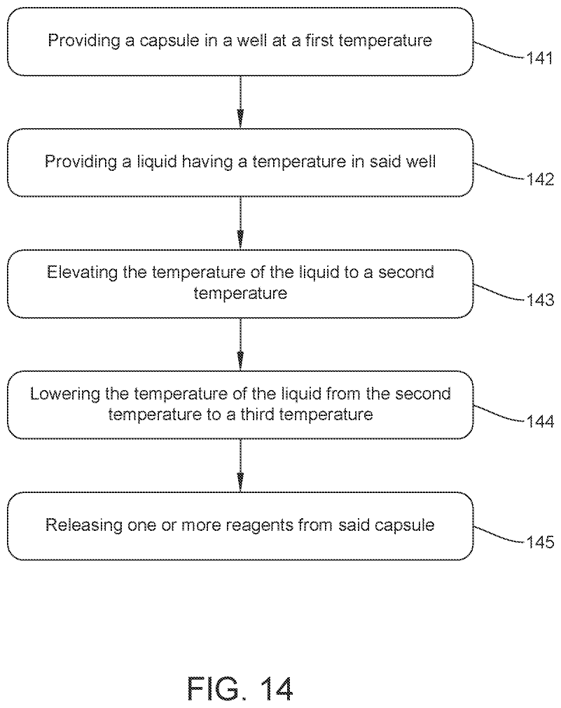

FIG. 14 is a flow chart describing one aspect described herein for a method. The method includes providing a capsule in a well at a first temperature 141. The method further includes providing a liquid having a temperature in said well 142. The method further includes elevating the temperature of the liquid to a second temperature 143. The method further includes lowering the temperature of the liquid from the second temperature to a third temperature 144. The method further includes releasing one or more reagents from said capsule 145.

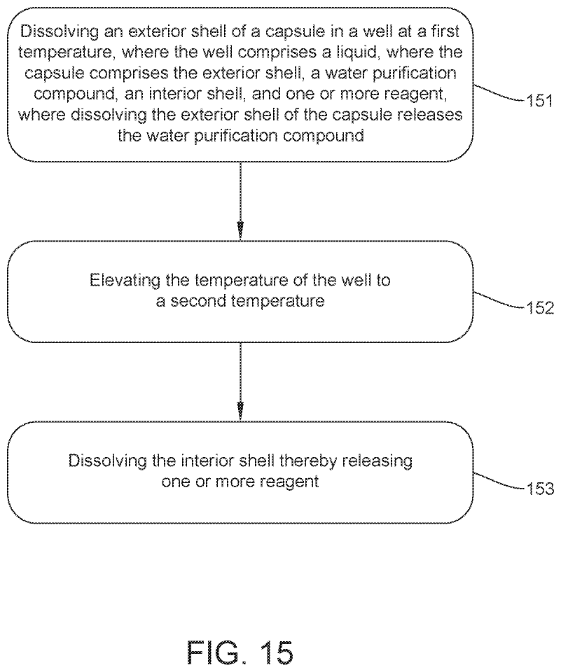

FIG. 15 is a flow chart describing one aspect described herein for a method. The method includes dissolving an exterior shell of a capsule in a well at a first temperature, where the well comprises a liquid, where the capsule comprises the exterior shell, a water purification compound, an interior shell, and one or more reagent, where dissolving the exterior shell of the capsule releases the water purification compound 151. The method further includes elevating the temperature of the well to a second temperature 152. The method further includes dissolving the interior shell thereby releasing one or more reagent 153.

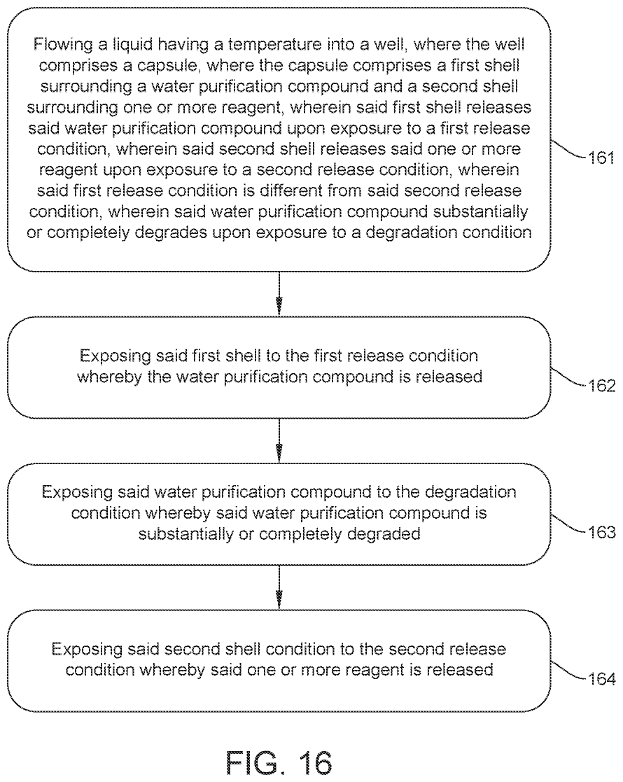

FIG. 16 is a flow chart describing one aspect described herein for a method. The method includes flowing a liquid having a temperature into a well, where the well comprises a capsule, where the capsule comprises a first shell surrounding a water purification compound and a second shell surrounding one or more reagent, wherein said first shell releases said water purification compound upon exposure to a first release condition, wherein said second shell releases said one or more reagent upon exposure to a second release condition, wherein said first release condition is different from said second release condition, wherein said water purification compound substantially or completely degrades upon exposure to a degradation condition 161. The method further includes exposing said first shell to the first release condition whereby the water purification compound is released 162. The method further includes exposing said water purification compound to the degradation condition whereby said water purification compound is substantially or completely degraded 163. The method further includes exposing said second shell condition to the second release condition whereby said one or more reagent is released 164.

Although preferred implementation have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

### IMPLEMENTATIONS

Various non-limiting Implementations of this disclosure are described in the text below:

[Implementation A] A composition comprising a shell surrounding an interior compartment, wherein said interior compartment comprises one or more reagent and wherein said shell releases said interior compartment when said shell is exposed to a first release condition, wherein said interior compartment releases said one or more reagent when said interior compartment is exposed to a second release condition, and wherein said first release condition is different from said second release condition.

[Implementation B] A composition according to Implementation [A] above, or according to other Implementations of the disclosure, wherein said interior compartment prevents release of said one or more reagent when said shell is exposed to said first release condition.

[Implementation C] A composition according to Implementation [A] or [B] above, or according to other Implementations of the disclosure, wherein said first release condition occurs before said second release condition.

[Implementation D] A composition according to any one of Implementations [A]-[C] above, or according to other Implementations of the disclosure, wherein said second release condition occurs after said first release condition.

[Implementation E] A composition according to any one of Implementations [A]-[D] above, or according to other Implementations of the disclosure, wherein said first release condition comprises a temperature-controlled release condition, a pH-controlled release condition, a time-controlled release condition, a position-controlled release condition, or any combination thereof.

[Implementation F] A composition according to any one of Implementations [A]-[E] above, or according to other Implementations of the disclosure, wherein said second release condition comprises a temperature-controlled release condition, a pH-controlled release condition, a time-controlled release condition, a position-controlled release condition, or any combination thereof.

[Implementation G] A composition according to any one of Implementations [A]-[F] above, or according to other Implementations of the disclosure, wherein either or both of the first and second release conditions comprise a change in temperature.

[Implementation H] A composition according to any one of Implementations [A]-[G] above, or according to other Implementations of the disclosure, wherein the change in temperature is to a temperature above about 25° C.

[Implementation I] A composition according to any one of Implementations [A]-[H] above, or according to other Implementations of the disclosure, wherein the change in temperature is to a temperature at or below about 25° C.

[Implementation J] A composition according to any one of Implementations [A]-[I] above, or according to other Implementations of the disclosure, wherein said shell releases said interior compartment when said shell is exposed to at least one additional shell release condition, wherein one or more of said at least one additional shell release condition is different from said first release condition.

[Implementation K] A composition according to any one of Implementations [A]-[J] above, or according to other Implementations of the disclosure, wherein said interior compartment prevents release of said one or more reagent when said shell is exposed to said at least one additional shell release condition.

[Implementation L] A composition according to any one of Implementations [A]-[K] above, or according to other Implementations of the disclosure, wherein said interior compartment releases said one or more reagent when said interior compartment is exposed to at least one additional interior compartment release condition, wherein one or more of said at least one additional interior compartment release condition is different from said second release condition.

[Implementation M] A composition according to any one of Implementations [A]-[L] above, or according to other Implementations of the disclosure, wherein said shell has a shell width and said interior compartment has an interior compartment width, and wherein said shell width is different from said interior compartment width.

[Implementation N] A composition according to any one of Implementations [A]-[M] above, or according to other Implementations of the disclosure, wherein said shell width is between about 1 micrometer and about 1,000 micrometers.

[Implementation O] A composition according to any one of Implementations [A]-[N] above, or according to other Implementations of the disclosure, wherein said interior compartment width is between about 1 micrometer and about 1,000 micrometers.

[Implementation P] A composition according to any one of Implementations [A]-[O] above, or according to other Implementations of the disclosure, wherein said shell comprises a water-soluble compound.

[Implementation Q] A composition according to any one of Implementations [A]-[P] above, or according to other Implementations of the disclosure, wherein said shell comprises one or more of polyvinyl alcohol, polyvinylpyrrolidone (PVP), carrageenan, gelatin, hydroxypropyl methylcellulose (HPMC), pullulan, starch film, benzoxaborole-poly(vinyl alcohol) (benzoxaborole-PVA), pectin, or any combination thereof.

[Implementation R] A composition according to any one of Implementations [A]-[Q] above, or according to other Implementations of the disclosure, wherein said one or more reagent is a sequencing reagent, a sample preparation reagent, a library preparation reagent, or a combination thereof.

[Implementation S] A composition according to any one of Implementations [A]-[R] above, or according to other Implementations of the disclosure, wherein said one or more reagent is selected from one or more enzyme, salt, surfactant, buffering agent, enzyme inhibitor, primer, nucleotide, organic osmolite, magnetic bead, molecular probe, crowding agent, small molecule, labelled-nucleotide, or any combination thereof.

[Implementation T] A composition according to any one of Implementations [A]-[S] above, or according to other Implementations of the disclosure, further comprising: a water purification compound.

[Implementation U] A composition according to any one of Implementations [A]-[T] above, or according to other Implementations of the disclosure, wherein said water purification compound comprises sodium dichloroisocyanurate, chlorine, chloramines, chlorine dioxide, polyaluminium chloride, aluminum sulfate, ferric sulfate, hydrogen peroxide, sodium hydroxide bromide, silver nanoparticles, iron, iodine, activated carbon, or any combination thereof.

[Implementation V] A composition according to any one of Implementations [A]-[U] above, or according to other Implementations of the disclosure, wherein said interior compartment comprises one or more dry reagent, one or more microsphere, one or more bead, one or more powder, one or more cake, one or more gel, one or more liquid, or any combination thereof.

[Implementation W] A composition according to any one of Implementations [A]-[V] above, or according to other Implementations of the disclosure, wherein said one or more reagent is lyophilised.

[Implementation X] A composition according to any one of Implementations [A]-[W] above, or according to other Implementations of the disclosure, wherein said interior compartment comprises a plurality of microspheres comprising a plurality of reagents.

[Implementation Y] A composition according to any one of Implementations [A]-[X] above, or according to other Implementations of the disclosure, wherein said interior compartment comprises a plurality of microspheres comprising one reagent.

[Implementation Z] A composition according comprising: a dissolvable first shell, and a dissolvable second shell, the second shell comprising one or more reagent.

[Implementation AA] A composition according to Implementation [Z] above, or according to other Implementations of the disclosure, wherein said first shell is an exterior shell.

[Implementation AB] A composition according to Implementation [Z] or [AA] above, or according to other Implementations of the disclosure, wherein said second shell is an interior shell.

[Implementation AC] A composition according to any one of Implementations [Z]-[AB] above, or according to other Implementations of the disclosure, wherein said first shell dissolves when said composition is exposed to a first release condition.

[Implementation AD] A composition according to any one of Implementations [Z]-[AC] above, or according to other Implementations of the disclosure, wherein said second shell prevents release of said one or more reagent when said composition is exposed to said first release condition.

[Implementation AE] A composition according to any one of Implementations [Z]-[AD] above, or according to other Implementations of the disclosure, wherein said second shell dissolves when exposed to a second release condition.

[Implementation AF] A composition according to any one of Implementations [Z]-[AE] above, or according to other Implementations of the disclosure, wherein said first release condition comprises a temperature-controlled release condition, a pH-controlled release condition, a time-controlled release condition, a position-controlled release condition, or any combination thereof.

[Implementation AG] A composition according to any one of Implementations [Z]-[AF] above, or according to other Implementations of the disclosure, wherein said second release condition comprises a temperature-controlled release condition, a pH-controlled release condition, a time-controlled release condition, a position-controlled release condition, or any combination thereof.

[Implementation AH] A composition according to any one of Implementations [Z]-[AG] above, or according to other Implementations of the disclosure, wherein either or both of the first and second release conditions comprise a change in temperature.

[Implementation AI] A composition according to any one of Implementations [Z]-[AH] above, or according to other Implementations of the disclosure, wherein the change in temperature is to a temperature above about 25° C.

[Implementation AJ] A composition according to any one of Implementations [Z]-[AI] above, or according to other Implementations of the disclosure, wherein the change in temperature is to a temperature at or below about 25° C.

[Implementation AK] A composition according to any one of Implementations [Z]-[AJ] above, or according to other Implementations of the disclosure, wherein said first shell dissolves when said first shell is exposed to at least one additional first shell release condition, wherein one or more of said at least one additional first shell release condition is different from said first release condition.

[Implementation AL] A composition according to any one of Implementations [Z]-[AK] above, or according to other Implementations of the disclosure, wherein said second shell prevents release of said one or more reagent when said second shell is exposed to said at least one additional first shell release condition.

[Implementation AM] A composition according to any one of Implementations [Z]-[AL] above, or according to other Implementations of the disclosure, wherein said second shell releases said one or more reagent when said second shell is exposed to at least one additional second shell release condition, wherein one or more of said at least one additional second shell release condition is different from said second release condition.

[Implementation AN] A composition according to any one of Implementations [Z]-[AM] above, or according to other Implementations of the disclosure, wherein said first shell has an first shell width and said second shell has a second shell width, and wherein said first shell width is different from said second shell width.

[Implementation AO] A composition according to any one of Implementations [Z]-[AN] above, or according to other Implementations of the disclosure, wherein said first shell width is between about 1 micrometer and about 1,000 micrometers.

[Implementation AP] A composition according to any one of Implementations [Z]-[AO] above, or according to other Implementations of the disclosure, wherein said second shell width is between about 1 micrometer and about 1,000 micrometers.

[Implementation AQ] A composition according to any one of Implementations [Z]-[AP] above, or according to other Implementations of the disclosure, wherein said first shell comprises a water-soluble compound.

[Implementation AR] A composition according to any one of Implementations [Z]-[AQ] above, or according to other Implementations of the disclosure, wherein said first shell comprises one or more of polyvinyl alcohol, polyvinylpyrrolidone (PVP), carrageenan, gelatin, hydroxypropyl methylcellulose (HPMC), pullulan, starch film, benzoxaborole-poly(vinyl alcohol) (benzoxaborole-PVA), pectin, or any combination thereof.

[Implementation AS] A composition according to any one of Implementations [Z]-[AR] above, or according to other Implementations of the disclosure, wherein said one or more reagent is a sequencing reagent, a sample preparation reagent, a library preparation reagent, or a combination thereof.

[Implementation AT] A composition according to any one of Implementations [Z]-[AS] above, or according to other Implementations of the disclosure, wherein said one or more reagent is selected from one or more enzyme, salt, surfactant, buffering agent, enzyme inhibitor, primer, nucleotide, organic osmolite, magnetic bead, molecular probe, crowding agent, small molecule, labelled-nucleotide, or any combination thereof.

[Implementation AU] A composition according to any one of Implementations [Z]-[AT] above, or according to other Implementations of the disclosure, further comprising: a water purification compound.

[Implementation AV] A composition according to any one of Implementations [Z]-[AU] above, or according to other Implementations of the disclosure, wherein said water purification compound comprises sodium dichloroisocyanurate, chlorine, chloramines, chlorine dioxide, polyaluminium chloride, aluminum sulfate, ferric sulfate, hydrogen peroxide, sodium hydroxide bromide, silver nanoparticles, iron, iodine, activated carbon, or any combination thereof.

[Implementation AW] A composition according to any one of Implementations [Z]-[AV] above, or according to other Implementations of the disclosure, wherein said second shell comprises one or more dry reagent, one or more microsphere, one or more bead, one or more powder, one or more cake, one or more gel, one or more liquid, or any combination thereof.

[Implementation AX] A composition according to any one of Implementations [Z]-[AW] above, or according to other Implementations of the disclosure, wherein said one or more reagent is lyophilised.

[Implementation AY] A composition according to any one of Implementations [Z]-[AX] above, or according to other Implementations of the disclosure, wherein said second shell comprises a plurality of microspheres comprising a plurality of reagents.

[Implementation AZ] A composition according to any one of Implementations [Z]-[AY] above, or according to other Implementations of the disclosure, wherein said second shell comprises a plurality of microspheres comprising one reagent.

[Implementation BA] A composition comprising: a dissolvable first shell; a dissolvable second shell, the second shell comprising one or more reagent; and a water purification compound.

[Implementation BB] A composition according to Implementation [BA] above, or according to other Implementations of the disclosure, wherein said water purification compound is in a position between said dissolvable first shell and said dissolvable second shell.

[Implementation BC] A composition according to Implementation [BA] or [BB] above, or according to other Implementations of the disclosure, wherein said water purification compound comprises sodium dichloroisocyanurate, chlorine, chloramines, chlorine dioxide, polyaluminium chloride, aluminum sulfate, ferric sulfate, hydrogen peroxide, sodium hydroxide bromide, silver nanoparticles, iron, iodine, activated carbon, or any combination thereof.

[Implementation BD] A composition according to any one of Implementations [BA]-[BC] above, or according to other Implementations of the disclosure, wherein said first shell is an exterior shell.

[Implementation BE] A composition according to any one of Implementations [BA]-[BD] above, or according to other Implementations of the disclosure, wherein said second shell is an interior shell.

[Implementation BF] A method for controlling release of one or more reagent, said method comprising: providing a composition comprising a shell surrounding an interior compartment, wherein said interior compartment comprises one or more reagent; exposing said composition to a first release condition to release said interior compartment; and exposing said interior compartment to a second release condition to release said one or more reagent, wherein said first release condition is different from said second release condition.

[Implementation BG] A method according to Implementation [BF] above, or according to other Implementations of the disclosure, wherein said interior compartment prevents release of said one or more reagent when said shell is exposed to said first release condition.

[Implementation BH] A method according to Implementation [BF] or [BG] above, or according to other Implementations of the disclosure, wherein said first release condition occurs before said second release condition.

[Implementation BI] A method according to any one of Implementations [BF]-[BH], wherein said second release condition occurs after said first release condition.

[Implementation BJ] A method according to any one of Implementations [BF]-[BI] above, or according to other Implementations of the disclosure, wherein said first release condition comprises a temperature-controlled release condition, a pH-controlled release condition, a time-controlled release condition, a position-controlled release condition, or any combination thereof.

[Implementation BK] A method according to any one of Implementations [BF]-[BJ] above, or according to other Implementations of the disclosure, wherein said second release condition comprises a temperature-controlled release condition, a pH-controlled release condition, a time-controlled release condition, a position-controlled release condition, or any combination thereof.

[Implementation BL] A method according to any one of Implementations [BF]-[BK] above, or according to other Implementations of the disclosure, wherein either or both of the first and second release conditions comprise a change in temperature.

[Implementation BM] A method according to any one of Implementations [BF]-[BL] above, or according to other Implementations of the disclosure, wherein the change in temperature is to a temperature above about 25° C.

[Implementation BN] A method according to any one of Implementations [BF]-[BM] above, or according to other Implementations of the disclosure, wherein the change in temperature is to a temperature at or below about 25° C.

[Implementation BO] A method according to any one of Implementations [BF]-[BN] above, or according to other Implementations of the disclosure, wherein said first release condition comprises a pH of between about 1.0 and about 10.0.

[Implementation BP] A method according to any one of Implementations [BF]-[BO] above, or according to other Implementations of the disclosure, wherein said second release condition comprises a pH of between about 1.0 and about 10.0.

[Implementation BQ] A method according to any one of Implementations [BF]-[BP], wherein said second release condition is effective to release a plurality of reagents, wherein the content of at least one reagent is different from the content of at least one other reagent.

[Implementation BR] A method according to any one of Implementations [BF]-[BQ] above, or according to other Implementations of the disclosure, wherein exposing said shell to said first release condition and exposing said interior compartment to said second release condition occurs sequentially.

[Implementation BS] A method according to any one of Implementations [BF]-[BR] above, or according to other Implementations of the disclosure, wherein said shell releases said interior compartment when said shell is exposed to at least one additional shell release condition, wherein one or more of said at least one additional shell release condition is different from said first release condition.

[Implementation BT] A method according to any one of Implementations [BF]-[BS] above, or according to other Implementations of the disclosure, wherein said interior compartment prevents release of said one or more reagent when said shell is exposed to said at least one additional shell release condition.

[Implementation BU] A method according to any one of Implementations [BF]-[BT] above, or according to other Implementations of the disclosure, wherein said interior compartment releases said one or more reagent when said interior compartment is exposed to at least one additional interior compartment release condition, wherein one or more of said at least one additional interior compartment release condition is different from said second release condition.

[Implementation BV] A method according to any one of Implementations [BF]-[BU] above, or according to other Implementations of the disclosure, wherein said shell has a shell width and said interior compartment has an interior compartment width, and wherein said shell width is different from said interior compartment width.

[Implementation BW] A method according to any one of Implementations [BF]-[BV] above, or according to other Implementations of the disclosure, wherein said shell width is between about 1 micrometer and about 1,000 micrometers.

[Implementation BX] A method according to any one of Implementations [BF]-[BW] above, or according to other Implementations of the disclosure, wherein said interior compartment width is between about 1 micrometer and about 1,000 micrometers.

[Implementation BY] A method according to any one of Implementations [BF]-[BX] above, or according to other Implementations of the disclosure, wherein said shell comprises a water-soluble compound.

[Implementation BZ] A method according to any one of Implementations [BF]-[BY] above, or according to other Implementations of the disclosure, wherein said shell comprises one or more of polyvinyl alcohol, polyvinylpyrrolidone (PVP), carrageenan, gelatin, hydroxypropyl methylcellulose (HPMC), pullulan, starch film, benzoxaborole-poly(vinyl alcohol) (benzoxaborole-PVA), pectin, or any combination thereof.

[Implementation CA] A method according to any one of Implementations [BF]-[BZ] above, or according to other Implementations of the disclosure, wherein said one or more reagent is a sequencing reagent, a sample preparation reagent, a library preparation reagent, or a combination thereof.

[Implementation CB] A method according to any one of Implementations [BF]-[CA] above, or according to other Implementations of the disclosure, wherein said one or more reagent is selected from one or more enzyme, salt, surfactant, buffering agent, enzyme inhibitor, primer, nucleotide, organic osmolite, magnetic bead, molecular probe, crowding agent, small molecule, labelled-nucleotide, or any combination thereof.

[Implementation CC] A method according to any one of Implementations [BF]-[CB] above, or according to other Implementations of the disclosure, further comprising: providing a water purification compound.

[Implementation CD] A method according to any one of Implementations [BF]-[CC] above, or according to other Implementations of the disclosure, wherein said water purification compound comprises sodium dichloroisocyanurate, chlorine, chloramines, chlorine dioxide, polyaluminium chloride, aluminum sulfate, ferric sulfate, hydrogen peroxide, sodium hydroxide bromide, silver nanoparticles, iron, iodine, activated carbon, or any combination thereof.

[Implementation CE] A method according to any one of Implementations [BF]-[CD] above, or according to other Implementations of the disclosure, wherein said interior compartment comprises one or more dry reagent, one or more microsphere, one or more bead, one or more powder, one or more cake, one or more gel, one or more liquid, or any combination thereof.

[Implementation CF] A method according to any one of Implementations [BF]-[CE] above, or according to other Implementations of the disclosure, wherein said one or more reagent is lyophilised.

[Implementation CG] A method according to any one of Implementations [BF]-[CF] above, or according to other Implementations of the disclosure, wherein said interior compartment comprises a plurality of microspheres comprising a plurality of reagents.

[Implementation CH] A method according to any one of Implementations [BF]-[CG] above, or according to other Implementations of the disclosure, wherein said interior compartment comprises a plurality of microspheres comprising one reagent.

[Implementation CI] A method for controlling release of one or more reagent, said method comprising: providing a composition comprising: a dissolvable first shell, and a dissolvable second shell, the second shell comprising one or more reagent; exposing said composition to a first release condition to dissolve said first shell; and exposing said composition to a second release condition to dissolve said second shell, wherein said first release condition is different from said second release condition.

[Implementation CJ] A method according to Implementation [CI] above, or according to other Implementations of the disclosure, wherein said second shell prevents release of said one or more reagent when said composition is exposed to said first release condition.

[Implementation CK] A method according to Implementation [CI] or [CJ] above, or according to other Implementations of the disclosure, wherein said first release condition comprises a temperature-controlled release condition, a pH-controlled release condition, a time-controlled release condition, a position-controlled release condition, or any combination thereof.

[Implementation CL] A method according to any one of Implementations [CI]-[CK] above, or according to other Implementations of the disclosure, wherein said second release condition comprises a temperature-controlled release condition, a pH-controlled release condition, a time-controlled release condition, a position-controlled release condition, or any combination thereof.

[Implementation CM] A method according to any one of Implementations [CI]-[CL] above, or according to other Implementations of the disclosure, wherein either or both of the first and second release conditions comprise a change in temperature.

[Implementation CN] A method according to any one of Implementations [CI]-[CM] above, or according to other Implementations of the disclosure, wherein the change in temperature is to a temperature above about 25° C.

[Implementation CO] A method according to any one of Implementations [CI]-[CN] above, or according to other Implementations of the disclosure, wherein the change in temperature is to a temperature at or below about 25° C.

[Implementation CP] A method according to any one of Implementations [CI]-[CO] above, or according to other Implementations of the disclosure, wherein said first shell dissolves when said first shell is exposed to at least one additional first shell release condition, wherein one or more of said at least one additional first shell release condition is different from said first release condition.

[Implementation CQ] A method according to any one of Implementations [CI]-[CP] above, or according to other Implementations of the disclosure, wherein said second shell prevents release of said one or more reagent when said second shell is exposed to said at least one additional second shell release condition.

[Implementation CR] A method according to any one of Implementations [CI]-[CQ] above, or according to other Implementations of the disclosure, wherein said second shell releases said one or more reagent when said second shell is exposed to at least one additional second shell release condition, wherein one or more of said at least one additional second shell release condition is different from said second release condition.

[Implementation CS] A method according to any one of Implementations [CI]-[CR] above, or according to other Implementations of the disclosure, wherein said first shell has a first shell width and said second shell has a second shell width, and wherein said first shell width is different from said second shell width.

[Implementation CT] A method according to any one of Implementations [CI]-[CS] above, or according to other Implementations of the disclosure, wherein said first shell width is between about 1 micrometer and about 1,000 micrometers.

[Implementation CU] A method according to any one of Implementations [CI]-[CT] above, or according to other Implementations of the disclosure, wherein said second shell width is between about 1 micrometer and about 1,000 micrometers.

[Implementation CV] A method according to any one of Implementations [CI]-[CU] above, or according to other Implementations of the disclosure, wherein said first shell comprises a water-soluble compound.

[Implementation CW] A method according to any one of Implementations [CI]-[CV] above, or according to other Implementations of the disclosure, wherein said first shell comprises one or more of polyvinyl alcohol, polyvinylpyrrolidone (PVP), carrageenan, gelatin, hydroxypropyl methylcellulose (HPMC), pullulan, starch film, benzoxaborole-poly(vinyl alcohol) (benzoxaborole-PVA), pectin, or any combination thereof.

[Implementation CX] A method according to any one of Implementations [CI]-[CW] above, or according to other Implementations of the disclosure, wherein said one or more reagent is a sequencing reagent, a sample preparation reagent, a library preparation reagent, or a combination thereof.

[Implementation CY] A method according to any one of Implementations [CI]-[CX] above, or according to other Implementations of the disclosure, wherein said one or more reagent is selected from one or more enzyme, salt, surfactant, buffering agent, enzyme inhibitor, primer, nucleotide, organic osmolite, magnetic bead, molecular probe, crowding agent, small molecule, labelled-nucleotide, or any combination thereof.

[Implementation CZ] A method according to any one of Implementations [CI]-[CY] above, or according to other Implementations of the disclosure, further comprising: providing a water purification compound.

[Implementation DA] A method according to any one of Implementations [CI]-[CZ] above, or according to other Implementations of the disclosure, wherein said water purification compound comprises sodium dichloroisocyanurate, chlorine, chloramines, chlorine dioxide, polyaluminium chloride, aluminum sulfate, ferric sulfate, hydrogen peroxide, sodium hydroxide bromide, silver nanoparticles, iron, iodine, activated carbon, or any combination thereof.

[Implementation DB] A method according to any one of Implementations [CI]-[DA] above, or according to other Implementations of the disclosure, wherein said second shell comprises one or more dry reagent, one or more microsphere, one or more bead, one or more powder, one or more cake, one or more gel, one or more liquid, or any combination thereof.

[Implementation DC] A method according to any one of Implementations [CI]-[DB] above, or according to other Implementations of the disclosure, wherein said one or more reagent is lyophilised.

[Implementation DD] A method according to any one of Implementations [CI]-[DC] above, or according to other Implementations of the disclosure, wherein said second shell comprises a plurality of microspheres comprising a plurality of reagents.

[Implementation DE] A method according to any one of Implementations [CI]-[DD] above, or according to other Implementations of the disclosure, wherein said second shell comprises a plurality of microspheres comprising one reagent.

[Implementation DF] A method according to any one of Implementations [CI]-[DE] above, or according to other Implementations of the disclosure, wherein said first shell is an exterior shell.

[Implementation DG] A method according to any one of Implementations [CI]-[DF] above, or according to other Implementations of the disclosure, wherein said second shell is an interior shell.

[Implementation DH] A method for controlling release of one or more reagent, said method comprising: providing a composition comprising: a dissolvable first shell, a dissolvable second shell, the second shell comprising one or more reagent, and a water purification compound; exposing said composition to a first release condition to dissolve said water purification compound; exposing said composition to a second condition to dissolve said first shell; and exposing said composition to a third release condition to dissolve said second shell, wherein said first release condition is different from said second release condition.

[Implementation DI] A method according to Implementation [DH] above, or according to other Implementations of the disclosure, wherein said water purification compound comprises sodium dichloroisocyanurate, chlorine, chloramines, chlorine dioxide, polyaluminium chloride, aluminum sulfate, ferric sulfate, hydrogen peroxide, sodium hydroxide bromide, silver nanoparticles, iron, iodine, activated carbon, or any combination thereof.

[Implementation DJ] A method according to Implementation [DH] or [DI] above, or according to other Implementations of the disclosure, wherein said first shell is an exterior shell.

[Implementation DK] A method according to any one of Implementations [DH]-[DJ] above, or according to other Implementations of the disclosure, wherein said second shell is an interior shell.

[Implementation DL] A method comprising: providing a capsule in a well at a first temperature; providing a liquid having a temperature in said well; elevating the temperature of the liquid to a second temperature; lowering the temperature of the liquid from the second temperature to a third temperature; and releasing one or more reagents from said capsule.

[Implementation DM] A method according to Implementation [DL] above, or according to other Implementations of the disclosure, wherein the capsule comprises the composition of any one of Implementations [A] through [Y].

[Implementation DN] A method according to Implementation [DL] or [DM] above, or according to other Implementations of the disclosure, wherein the capsule comprises the composition of any one of Implementations [Z] through [AZ].

[Implementation DO] A method according to any one of Implementations [DL]-[DN] above, or according to other Implementations of the disclosure, wherein the capsule comprises the composition of Implementation [BA] through [BE].

[Implementation DP] A method according to any one of Implementations [DL]-[DO] above, or according to other Implementations of the disclosure, wherein said second temperature is above about 25° C.

[Implementation DQ] A method according to any one of Implementations [DL]-[DP] above, or according to other Implementations of the disclosure, wherein said third temperature is at or below about 25° C.

[Implementation DR] A method according to any one of Implementations [DL]-[DQ] above, or according to other Implementations of the disclosure, further comprising: providing a water purification compound.

[Implementation DS] A method according to any one of Implementations [DL]-[DR] above, or according to other Implementations of the disclosure, wherein said water purification compound comprises sodium dichloroisocyanurate, chlorine, chloramines, chlorine dioxide, polyaluminium chloride, aluminum sulfate, ferric sulfate, hydrogen peroxide, sodium hydroxide bromide, silver nanoparticles, iron, iodine, activated carbon, or any combination thereof.

[Implementation DT] A method according to any one of Implementations [DL]-[DS] above, or according to other Implementations of the disclosure, wherein said capsule comprises a water-soluble compound.

[Implementation DU] A method according to any one of Implementations [DL]-[DT], wherein said capsule comprises one or more of polyvinyl alcohol, polyvinylpyrrolidone (PVP), carrageenan, gelatin, hydroxypropyl methylcellulose (HPMC), pullulan, starch film, benzoxaborole-poly(vinyl alcohol) (benzoxaborole-PVA), pectin, or any combination thereof.

[Implementation DV] A method according to any one of Implementations [DL]-[DU] above, or according to other Implementations of the disclosure, wherein said one or more reagent is a sequencing reagent, a sample preparation reagent, a library preparation reagent, or a combination thereof.

[Implementation DW] A method according to any one of Implementations [DL]-[DV] above, or according to other Implementations of the disclosure, wherein said one or more reagent is selected from one or more enzyme, salt, surfactant, buffering agent, enzyme inhibitor, primer, nucleotide, organic osmolite, magnetic bead, molecular probe, crowding agent, small molecule, labelled-nucleotide, or any combination thereof.

[Implementation DX] A method according to any one of Implementations [DL]-[DW] above, or according to other Implementations of the disclosure, wherein said interior compartment comprises one or more dry reagent, one or more microsphere, one or more bead, one or more powder, one or more cake, one or more gel, one or more liquid, or any combination thereof.

[Implementation DY] A method according to any one of Implementations [DL]-[DX] above, or according to other Implementations of the disclosure, wherein said one or more reagent is lyophilised.

[Implementation DZ] A method according to any one of Implementations [DL]-[DY] above, or according to other Implementations of the disclosure, wherein said interior compartment comprises a plurality of microspheres comprising a plurality of reagents.

[Implementation EA] A method according to any one of Implementations [DL]-[DZ] above, or according to other Implementations of the disclosure, wherein said interior compartment comprises a plurality of microspheres comprising one reagent.

[Implementation EB] A method according to any one of Implementations [DL]-[EA] above, or according to other Implementations of the disclosure, wherein the first temperature is different from the third temperature.

[Implementation EC] A method according to any one of Implementations [DL]-[EB] above, or according to other Implementations of the disclosure, wherein the first temperature is the same as the third temperature.

[Implementation ED] A method comprising: dissolving an exterior shell of a capsule in a well at a first temperature, where the well comprises a liquid, where the capsule comprises the exterior shell, a water purification compound, an interior shell, and one or more reagent, where dissolving the exterior shell of the capsule releases the water purification compound; elevating the temperature of the well to a second temperature; and dissolving the interior shell thereby releasing one or more reagent.

[Implementation EE] A method according to Implementation [ED] above, or according to other Implementations of the disclosure, wherein dissolving the exterior shell of the capsule in the well comprises flowing the liquid into the well.

[Implementation EF] A method according to Implementation [ED] or [EE] above, or according to other Implementations of the disclosure, wherein dissolving the interior shell comprises raising the pH of the liquid above 7.0.

[Implementation EG] A method according to any one of Implementations [ED]-[EF] above, or according to other Implementations of the disclosure, wherein dissolving the interior shell comprises lowering the pH of the liquid below 7.0.

[Implementation EH] A method according to any one of Implementations [ED]-[EG] above, or according to other Implementations of the disclosure, wherein the interior shell is dissolved by the second temperature.

[Implementation EI] A method according to any one of Implementations [ED]-[EH] above, or according to other Implementations of the disclosure, wherein the interior shell is dissolved after a minimum time period.

[Implementation EJ] A method according to any one of Implementations [ED]-[EI] above, or according to other Implementations of the disclosure, wherein the minimum time period is 5 minutes.

[Implementation EK] A method according to any one of Implementations [ED]-[EJ] above, or according to other Implementations of the disclosure, wherein said second temperature is above about 25° C.

[Implementation EL] A method according to any one of Implementations [ED]-[EK] above, or according to other Implementations of the disclosure, further comprising: lowering said second temperature to a third temperature.

[Implementation EM] A method according to any one of Implementations [ED]-[EL] above, or according to other Implementations of the disclosure, wherein said water purification compound comprises sodium dichloroisocyanurate, chlorine, chloramines, chlorine dioxide, polyaluminium chloride, aluminum sulfate, ferric sulfate, hydrogen peroxide, sodium hydroxide bromide, silver nanoparticles, iron, iodine, activated carbon, or any combination thereof.

[Implementation EN] A method according to any one of Implementations [ED]-[EM] above, or according to other Implementations of the disclosure, wherein said shell comprises a water-soluble compound.

[Implementation EO] A method according to any one of Implementations [ED]-[EN] above, or according to other Implementations of the disclosure, wherein said shell comprises one or more of polyvinyl alcohol, polyvinylpyrrolidone (PVP), carrageenan, gelatin, hydroxypropyl methylcellulose (HPMC), pullulan, starch film, benzoxaborole-poly(vinyl alcohol) (benzoxaborole-PVA), pectin, or any combination thereof.

[Implementation EP] A method according to any one of Implementations [ED]-[EO] above, or according to other Implementations of the disclosure, wherein said one or more reagent is a sequencing reagent, a sample preparation reagent, a library preparation reagent, or a combination thereof.

[Implementation EQ] A method according to any one of Implementations [ED]-[EP] above, or according to other Implementations of the disclosure, wherein said one or more reagent is selected from one or more enzyme, salt, surfactant, buffering agent, enzyme inhibitor, primer, nucleotide, organic osmolite, magnetic bead, molecular probe, crowding agent, small molecule, labelled-nucleotide, or any combination thereof.

[Implementation ER] A method according to any one of Implementations [ED]-[EQ] above, or according to other Implementations of the disclosure, wherein said interior compartment comprises one or more dry reagent, one or more microsphere, one or more bead, one or more powder, one or more cake, one or more gel, one or more liquid, or any combination thereof.

[Implementation ES] A method according to any one of Implementations [ED]-[ER] above, or according to other Implementations of the disclosure, wherein said one or more reagent is lyophilised.

[Implementation ET] A method according to any one of Implementations [ED]-[ES] above, or according to other Implementations of the disclosure, wherein said interior compartment comprises a plurality of microspheres comprising a plurality of reagents.

[Implementation EU] A method according to any one of Implementations [ED]-[ET] above, or according to other Implementations of the disclosure, wherein said interior compartment comprises a plurality of microspheres comprising one reagent.

[Implementation EV] A cartridge comprising: a reagent reservoir, wherein the reagent reservoir comprises a composition, said composition comprising: a shell surrounding an interior compartment, wherein said interior compartment comprises one or more reagent and wherein said shell releases said interior compartment when said shell is exposed to a first release condition, wherein said interior compartment releases said one or more reagent when said interior compartment is exposed to a second release condition, and wherein said first release condition is different from said second release condition.

[Implementation EW] A cartridge according to Implementation [EV] above, or according to other Implementations of the disclosure, wherein said cartridge comprises a water purification compound.

[Implementation EX] A cartridge according to Implementation [EW] above, or according to other Implementations of the disclosure, wherein said water purification compound comprises sodium dichloroisocyanurate, chlorine, chloramines, chlorine dioxide, polyaluminium chloride, aluminum sulfate, ferric sulfate, hydrogen peroxide, sodium hydroxide bromide, silver nanoparticles, iron, iodine, activated carbon, or any combination thereof.

[Implementation EY] A cartridge according to Implementation [EX] above, or according to other Implementations of the disclosure, wherein the first release condition is exposure to a liquid.

[Implementation EZ] A cartridge according to Implementation [EY] above, or according to other Implementations of the disclosure, wherein the second release condition is exposure to a temperature above about 25° C.

[Implementation FA] A cartridge comprising: a reagent reservoir, wherein the reagent reservoir comprises a composition, said composition comprising: a dissolvable first shell, and a dissolvable second shell, the second shell comprising one or more reagent.

[Implementation FB] A cartridge according to Implementation [FA] above, or according to other Implementations of the disclosure, wherein said cartridge comprises a water purification compound.

[Implementation FC] A cartridge according to Implementation [FA] or [FB] above, or according to other Implementations of the disclosure, wherein said water purification compound comprises sodium dichloroisocyanurate, chlorine, chloramines, chlorine dioxide, polyaluminium chloride, aluminum sulfate, ferric sulfate, hydrogen peroxide, sodium hydroxide bromide, silver nanoparticles, iron, iodine, activated carbon, or any combination thereof.

[Implementation FD] A cartridge according to any one of Implementations [FA]-[FC] above, or according to other Implementations of the disclosure, wherein the first release condition is exposure to a liquid.

[Implementation FE] A cartridge according to any one of Implementations [FA]-[FD] above, or according to other Implementations of the disclosure, wherein the second release condition is exposure to a temperature above about 25° C.

[Implementation FF] A cartridge according to any one of Implementations [FA]-[FE] above, or according to other Implementations of the disclosure, wherein said first shell is an exterior shell.

[Implementation FG] A cartridge according to any one of Implementations [FA]-[FF] above, or according to other Implementations of the disclosure, wherein said second shell is an interior shell.

[Implementation FH] A system for controlling release of one or more reagent comprising: a well; a composition comprising: a shell surrounding an interior compartment, wherein said interior compartment comprises one or more reagent, and wherein said shell releases said interior compartment when said shell is exposed to a first release condition, wherein said interior compartment releases said one or more reagent when said interior compartment is exposed to a second release condition, and wherein said first release condition is different from said second release condition; and a liquid.

[Implementation FI] A system according to Implementation [FH] above, or according to other Implementations of the disclosure, wherein said liquid is in said well.

[Implementation FJ] A system according to Implementation [FH] or [FI] above, or according to other Implementations of the disclosure, wherein said composition is in said well.

[Implementation FK] A system according to any one of Implementations [FH]-[FJ] above, or according to other Implementations of the disclosure, further comprising: a temperature controller on said well.

[Implementation FL] A system according to any one of Implementations [FH]-[FK] above, or according to other Implementations of the disclosure, further comprising: a water purification compound.

[Implementation FM] A system according to any one of Implementations [FH]-[FL] above, or according to other Implementations of the disclosure, wherein said water purification compound comprises sodium dichloroisocyanurate, chlorine, chloramines, chlorine dioxide, polyaluminium chloride, aluminum sulfate, ferric sulfate, hydrogen peroxide, sodium hydroxide bromide, silver nanoparticles, iron, iodine, activated carbon, or any combination thereof.

[Implementation FN] A system for controlling release of one or more reagent comprising: a well; a composition comprising: a dissolvable first shell, and a dissolvable second shell, the second shell comprising one or more reagent; and a liquid.

[Implementation FO] A system according to Implementation [FN] above, or according to other Implementations of the disclosure, wherein said liquid is in said well.

[Implementation FP] A system according to Implementation [FN] or [FO] above, or according to other Implementations of the disclosure, wherein said composition is in said well.

[Implementation FQ] A system according to any one of Implementations [FN]-[FP] above, or according to other Implementations of the disclosure, further comprising: a temperature controller on said well.

[Implementation FR] A system according to any one of Implementations [FN]-[FQ] above, or according to other Implementations of the disclosure, further comprising: a water purification compound.

[Implementation FS] A system according to any one of Implementations [FN]-[FR] above, or according to other Implementations of the disclosure, wherein said water purification compound comprises sodium dichloroisocyanurate, chlorine, chloramines, chlorine dioxide, polyaluminium chloride, aluminum sulfate, ferric sulfate, hydrogen peroxide, sodium hydroxide bromide, silver nanoparticles, iron, iodine, activated carbon, or any combination thereof.

[Implementation FT] A system according to any one of Implementations [FN]-[FS] above, or according to other Implementations of the disclosure, wherein said first shell is an exterior shell.

[Implementation FU] A system according to any one of Implementations [FN]-[FT] above, or according to other Implementations of the disclosure, wherein said second shell is an interior shell.

[Implementation FV] A method comprising: flowing a liquid having a temperature into a well, where the well comprises a capsule, where the capsule comprises a first shell surrounding a water purification compound and a second shell surrounding one or more reagent, wherein said first shell releases said water purification compound upon exposure to a first release condition, wherein said second shell releases said one or more reagent upon exposure to a second release condition, wherein said first release condition is different from said second release condition, wherein said water purification compound substantially or completely degrades upon exposure to a degradation condition; exposing said first shell to the first release condition whereby the water purification compound is released; exposing said water purification compound to the degradation condition whereby said water purification compound is substantially or completely degraded; and exposing said second shell condition to the second release condition whereby said one or more reagent is released.

[Implementation FW] A method according to Implementation [FV] above, or according to other Implementations of the disclosure, wherein the first release condition is exposure to the liquid.

[Implementation FX] A method according to Implementation [FV] or [FW] above, or according to other Implementations of the disclosure, wherein the degradation condition is an elevated temperature of the liquid.

[Implementation FY] A method according to any one of Implementations [FV]-[FX] above, or according to other Implementations of the disclosure, wherein the elevated temperature is greater than or equal to about 25° C.

[Implementation FZ] A method according to any one of Implementations [FV]-[FY] above, or according to other Implementations of the disclosure, wherein the degradation condition is the same as the second release condition.

[Implementation GA] A method according to any one of Implementations [FV]-[FZ] above, or according to other Implementations of the disclosure, wherein flowing a liquid, exposing said first shell to the first release condition, and exposing said water purification compound to the degradation condition are performed in order.

[Implementation GB] A method according to any one of Implementations [FV]-[GA] above, or according to other Implementations of the disclosure, wherein flowing a liquid, exposing said first shell to the first release condition, exposing said water purification compound to the degradation condition, and exposing said second shell condition to the second release condition are performed in order.

1-184. (canceled)

185. A composition comprising:
a shell surrounding an interior compartment, said interior compartment comprising one or more reagent,
wherein said shell releases said interior compartment when said shell is exposed to a first release condition, and said interior compartment prevents release of said one or more reagent when said shell is exposed to said first release condition,
wherein said interior compartment releases said one or more reagent when said interior compartment is exposed to a second release condition, and wherein said first release condition is different from said second release condition.

186. The composition of claim 185, wherein said first release condition and said second release condition are each independently selected from a temperature-controlled release condition, a pH-controlled release condition, a time-controlled release condition, a position-controlled release condition, or any combination thereof.

187. The composition of claim 185, wherein either or both of the first and second release conditions comprise a change in temperature, wherein the change in temperature is to a temperature (i) above about 25° C. or (ii) at or below about 25° C.

188. The composition of claim 185, wherein either or both of
(i) said shell releases said interior compartment when said shell is exposed to at least one additional shell release condition, wherein one or more of said at least one additional shell release condition is different from said first release condition, and
(ii) said interior compartment releases said one or more reagent when said interior compartment is exposed to at least one additional interior compartment release condition, wherein one or more of said at least one additional interior compartment release condition is different from said second release condition.

189. The composition of claim 185, wherein said shell has a shell width between about 1 micrometer and about 1,000 micrometers and said interior compartment has an interior compartment width between about 1 micrometer and about 1,000 micrometers, and wherein said shell width is different from said interior compartment width.

190. The composition of claim 185, wherein said shell comprises a water-soluble compound.

191. The composition of claim 185, wherein said one or more reagent is a sequencing reagent, a sample preparation reagent, a library preparation reagent, or any combination thereof.

192. The composition of claim 185, further comprising a water purification compound, wherein the water purification compound comprises sodium dichloroisocyanurate, chlorine, chloramines, chlorine dioxide, polyaluminium chloride, aluminum sulfate, ferric sulfate, hydrogen peroxide, sodium hydroxide bromide, silver nanoparticles, iron, iodine, activated carbon, or any combination thereof.

193. The composition of claim 185, wherein said interior compartment comprises one or more dry reagent, one or more microsphere, one or more bead, one or more powder, one or more cake, one or more gel, one or more liquid, or any combination thereof.

194. The composition of claim 185, wherein said one or more reagent is lyophilised.

195. A method for controlling release of one or more reagent, said method comprising:
exposing a composition comprising a shell surrounding an interior compartment to a first release condition to release said interior compartment, wherein said interior compartment comprises one or more reagent; and
exposing said interior compartment to a second release condition to release said one or more reagent, wherein said first release condition is different from said second release condition.

196. The method of claim 195, wherein said first release condition and said second release condition are each independently selected from a temperature-controlled release condition, a pH-controlled release condition, a time-controlled release condition, a position-controlled release condition, or any combination thereof.

197. The method of claim 195, wherein said shell has a shell width between about 1 micrometer and about 1,000 micrometers and said interior compartment has an interior compartment width between about 1 micrometer and about 1,000 micrometers, and wherein said shell width is different from said interior compartment width.

198. The method of claim 195, wherein said one or more reagent is a sequencing reagent, a sample preparation reagent, a library preparation reagent, or a combination thereof.

199. The method of claim 195, wherein either or both of said shell and said interior compartment further comprises a water purification compound, wherein said water purification compound comprises sodium dichloroisocyanurate, chlorine, chloramines, chlorine dioxide, polyaluminium chloride, aluminum sulfate, ferric sulfate, hydrogen peroxide, sodium hydroxide bromide, silver nanoparticles, iron, iodine, activated carbon, or any combination thereof.

200. The method of claim 199, wherein said water purification compound substantially or completely degrades upon exposure to a degradation condition, wherein said degradation condition comprises a change in temperature to a temperature at or above 25° C.

201. The method of claim 195, wherein said one or more reagent is lyophilised.

202. A system for controlling release of one or more reagent comprising:
a well;
a composition comprising: a shell surrounding an interior compartment, wherein said interior compartment comprises one or more reagent, and wherein said shell releases said interior compartment when said shell is exposed to a first release condition, wherein said interior compartment releases said one or more reagent when said interior compartment is exposed to a second release condition, and wherein said first release condition is different from said second release condition; and
a liquid.

203. The system of claim 202, wherein either or both of (i) said liquid is in said well and (ii) said composition is in said well.

204. The system of claim 202, further comprising either or both of (i) a temperature controller on said well and (ii) a water purification compound in said composition, wherein said water purification compound comprises sodium dichloroisocyanurate, chlorine, chloramines, chlorine dioxide, polyaluminium chloride, aluminum sulfate, ferric sulfate, hydrogen peroxide, sodium hydroxide bromide, silver nanoparticles, iron, iodine, activated carbon, or any combination thereof.
